SEQUENCE LISTING (2 sequences)

SEQ ID NO: 1 - DNA, 100 bp, Artificial Sequence
  Primer
cggccaagcttactcggatccagagtttgcaaaatatacaggggattatatataatggaaaacaagaaaggaaaataggaggtttatatggaagacgcca

SEQ ID NO: 2 - DNA, 20 bp, Artificial Sequence
  Primer
gtcatcgtcgggaagacctg

(19) United States
(12) Patent Application Publication
(10) Pub. No.: US 2008/0306040 A1
(43) Pub. Date: Dec. 11, 2008

(54) Antibacterial amide macrocycles VII

(76) Inventors: Rainer Endermann, Wuppertal (DE); Kerstin Ehlert, Velbert (DE); Martin Michels, Koeln (DE); Stefan Weigand, Penzberg (DE); Guido Schiffer, Wuppertal (DE)

Correspondence Address: MORRISON & FOERSTER LLP, 12531 HIGH BLUFF DRIVE, SUITE 100, SAN DIEGO, CA, 92130-2040, US

(21) Appl. No.: 12/008,662

(22) Filed: Jan. 11, 2008

Related U.S. Application Data
(63) Continuation of application No. PCT/EP2006/006770, filed on Jul. 11, 2006.

(30) Foreign Application Priority Data
Jul. 14, 2005 (DE) DE102005032781.8

(51) Int. Cl. A61K 31/395; C07D 245/04; A61P 31/04

(52) U.S. Cl. 514/183; 540/460

(57) ABSTRACT

The invention relates to antibacterial amide macrocycles and methods for their preparation, their use for the treatment and/or prophylaxis of diseases as well as their use for the production of medicaments for the treatment and/or prophylaxis of diseases, especially bacterial infections.

### CROSS REFERENCE TO RELATED APPLICATIONS

This application is a continuation of pending international application PCT/EP2006/006770, filed Jul. 11, 2006, designating U.S., which claims priority from German patent application DE 10 2005 032 781.8, filed Jul. 14, 2005. The contents of the above-referenced applications are incorporated herein by this reference in their entirety.

### BACKGROUND OF THE INVENTION

The invention relates to antibacterial amide macrocycles and methods for their preparation, their use for the treatment and/or prophylaxis of diseases, as well as their use for the production of medicaments for the treatment and/or prophylaxis of diseases, in particular of bacterial infections.

WO 03/106480, WO 04/012816, WO 05/033129 and WO 05/058943 describe macrocycles of the biphenomycin B type which have antibacterial activity and have amide and ester substituents, respectively.

U.S. Pat. No. 3,452,136; thesis of R. U. Meyer, Stuttgart University, Germany 1991; thesis of V. Leitenberger, Stuttgart University, Germany 1991; Synthesis (1992), (10), 1025-1030; J. Chem. Soc., Perkin Trans. 1 (1992), (1), 123-130, J. Chem. Soc., Chem. Commun. (1991), (10), 744; Synthesis (1991), (5), 409-413; J. Chem. Soc., Chem. Commun. (1991), (5), 275-277; J. Antibiot. (1985), 38(11), 1462-1468; J. Antibiot. (1985), 38(11), 1453-1461 describe the natural product biphenomycin B as having antibacterial activity. Some steps in the synthesis of biphenomycin B are described in Synlett (2003), 4, 522-526 and Org. Lett. (2005), (7), 2981-2984.

Chirality (1995), 7(4), 181-192; J. Antibiot. (1991), 44(6), 674-677; J. Am. Chem. Soc. (1989), 111(19), 7323-7327; J. Am. Chem. Soc. (1989), 111(19), 7328-7333; J. Org. Chem. (1987), 52(24), 5435-5437; Anal. Biochem. (1987), 165(1), 108-113; J. Org. Chem. (1985), 50(8), 1341-1342; J. Antibiot. (1993), 46(3), C-2; J. Antibiot. (1993), 46(1), 135-140; Synthesis (1992), (12), 1248-1254; Appl. Environ. Microbiol. (1992), 58(12), 3879-8; J. Chem. Soc., Chem. Commun. (1992), (13), 951-953 describe a structurally related natural product, biphenomycin A, which has a further substitution with a hydroxy group on the macrocycle.

The natural products in terms of their properties do not comply with the requirements for antibacterial medicaments. Although structurally different agents with antibacterial activity are available on the market, the development of resistance is a regular possibility. Novel agents for a good and more effective therapy are therefore desirable.

### SUMMARY OF THE INVENTION

One object of the present invention is therefore to provide novel and alternative compounds with the same or improved antibacterial activity for the treatment of bacterial diseases in humans and animals.

It has surprisingly been found that certain derivatives of these natural products wherein the carboxy group of the natural product is replaced with a methylene amide group which comprises a basic group have antibacterial activity.

In addition, the derivatives show an antibacterial activity against biphenomycin-resistant S. aureus strains (RN4220Bi^{R} and T17) and a spontaneous resistance rate for S. aureus wild-type strains and biphenomycin-resistant S. aureus strains which is better than or the same as biphenomycin and the biphenomycin derivatives known from the prior art.

The invention relates to compounds of formula

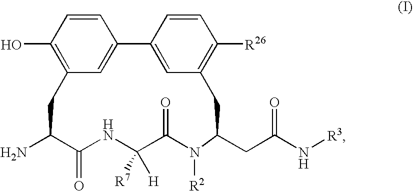

in which

R²⁶ represents hydrogen, halogen, hydroxy or methyl,

R⁷ represents a group of formula

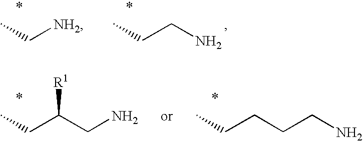

whereby

R¹ represents hydrogen or hydroxy,

* is the linkage site to the carbon atom,

R² represents hydrogen or methyl,

R³ represents a group of formula

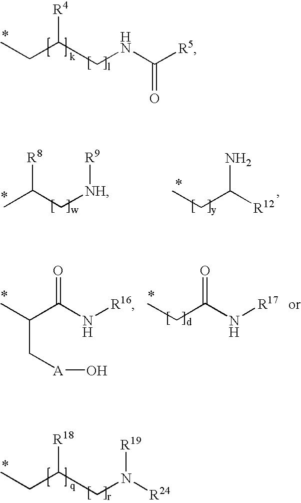

whereby

* is the linkage site to the nitrogen atom,

A represents a bond or phenyl,

R⁴ represents hydrogen, amino or hydroxy,

R⁵ represents a group of formula

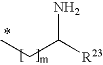

wherein

* is the linkage site to the carbon atom,

R²³ represents hydrogen or a group of formula *-(CH₂)ₙ—OH or *-(CH₂)ₙ—NH₂,

wherein

* is the linkage site to the carbon atom,
n and o independently of one another are a number 1, 2, 3 or 4,

m represents a number 0 or 1,

R⁸ and R¹² independently of one another represent a group of formula *-CONHR¹⁴ or *-CH₂CONHR¹⁵,
wherein

* is the linkage site to the carbon atom,

R¹⁴ and R¹⁵ independently of one another represent a group of formula

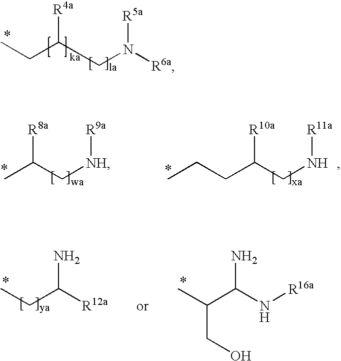

wherein

* is the linkage site to the nitrogen atom,

R^{4a} represents hydrogen, amino or hydroxy,

R^{5a} represents hydrogen, methyl or aminoethyl,

R^{6a} represents hydrogen or aminoethyl, or

R^{5a} and R^{6a} together with the nitrogen atom to which they are bonded form a piperazine ring,

R^{8a} and R^{12a} independently of one another represent *-(CH₂)_{Z1a}—OH, *-(CH₂)_{Z2a}—NHR^{13a}, *-CONHR^{4a} or *-CH₂CONHR^{15a},
wherein
   * is the linkage site to the carbon atom,

Z1a and Z2a independently of one another are a number 1, 2 or 3,

R^{13a} represents hydrogen or methyl,

and

R^{14a} and R^{15a} independently of one another represent a group of formula

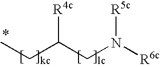

wherein

* is the linkage site to the nitrogen atom,

R^{4c} represents hydrogen, amino or hydroxy,

R^{5c} represents hydrogen, methyl or aminoethyl,

R^{6c} represents hydrogen or aminoethyl,
kc is a number 0 or 1,
and
lc is a number 1, 2, 3 or 4,

R^{9a} and R^{11a} independently of one another represent hydrogen or methyl,

R^{10a} represents amino or hydroxy,

R^{16a} represents a group of formula

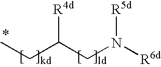

wherein

* is the linkage site to the nitrogen atom,

R^{4d} represents hydrogen, amino or hydroxy,

R^{5d} represents hydrogen, methyl or aminoethyl,

R^{6d} represents hydrogen or aminoethyl,
kd is a number 0 or 1,
and
ld is a number 1, 2, 3 or 4,

ka is a number 0 or 1,

and

la, wa, xa and ya independently of one another are a number 1, 2, 3 or 4,

R⁹ represents hydrogen, methyl, *-C(NH2)=NH or a group of formula

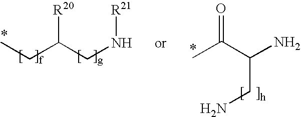

wherein

* is the linkage site to the nitrogen atom,

R²⁰ represents hydrogen or *-(CH₂)ᵢ—NHR²²
wherein

R²² represents hydrogen or methyl,

and
i is a number 1, 2 or 3,

R²¹ represents hydrogen or methyl,
f is a number 0, 1, 2 or 3,
g is a number 1, 2 or 3,
and
h is a number 1, 2, 3 or 4,

R¹⁶ and R¹⁷ independently of one another represent a group of formula

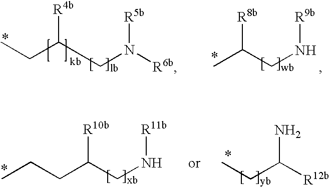

wherein

* is the linkage site to the nitrogen atom,

R^{4b} represents hydrogen, amino or hydroxy,

R^{5b} represents hydrogen, methyl or aminoethyl,

R^{6b} represents hydrogen or aminoethyl,

or

R^{5b} and R^{6b} together with the nitrogen atom to which they are bonded form a piperazine ring,

R^{8b} and R^{12b} independently of one another represent *-(CH₂)_{Z1b}—OH, *-(CH₂)_{z2b}—NHR^{13b}, *-CONHR^{14b} or *-CH₂CONHR^{15b},
wherein
   * is the linkage site to the carbon atom,

R^{13b} represents hydrogen or methyl,

and

Z1b and Z2b independently of one another are a number 1, 2 or 3,

and

R^{14b} and R^{15b} independently of one another represent a group of formula

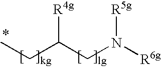

wherein

* is the linkage site to the nitrogen atom,

R^{4g} represents hydrogen, amino or hydroxy,

R^{5g} represents hydrogen, methyl or aminoethyl,

R^{6g} represents hydrogen or aminoethyl,
kg is a number 0 or 1,
and
lg is a number 1, 2, 3 or 4,

R^{9b} and R^{11b} independently of one another represent hydrogen or methyl,

R^{10b} represents amino or hydroxy,

kb is a number 0 or 1,

lb, wb, xb and yb independently of one another are a number 1, 2, 3 or 4,

R¹⁸ represents hydrogen, amino or hydroxy,

R¹⁹ represents hydrogen, methyl or a group of formula

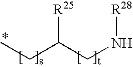

wherein

* is the linkage site to the nitrogen atom,

R²⁵ represents hydrogen or *-(CH₂)ᵤ—NHR²⁹,
wherein

R²⁹ represents hydrogen or methyl and
u is a number 1, 2 or 3,

R²⁸ represents hydrogen or methyl,

is a number 0, 1, 2 or 3,

and

t is a number 1, 2 or 3,

R²⁴ represents hydrogen or aminoethyl,

d is a number 1, 2 or 3,

k and q independently of one another are a number 0 or 1,

l, r, w and y independently of one another are a number 1, 2, 3 or 4,

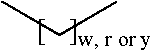

independently of one another may when w, r or y equals 3 carry a hydroxy group, and their salts, their solvates and the solvates of their salts.

Compounds of the invention are the compounds of formula (I) and the salts, solvates and solvates of the salts thereof, as well as the compounds which are encompassed by formula (I) and are mentioned hereinafter as exemplary embodiment(s), and the salts, solvates and solvates of the salts thereof, insofar as the compounds which are encompassed by formula (I) and are mentioned hereinafter are not already salts, solvates and solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure constituents can be isolated from such mixtures of enantiomers and/or diastereomers in a known way by known methods such as chromatography on a chiral phase or crystallization using chiral amines or chiral acids.

The invention also relates, depending on the structure of the compounds, to tautomers of the compounds.

Salts preferred for the purposes of the invention are physiologically acceptable salts of the compounds of the invention.

Physiologically acceptable salts of the compounds (I) include acid addition salts of mineral acids, carboxylic acids and sulfonic acids, e.g., salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid, trifluoroacetic acid and benzoic acid.

Physiologically acceptable salts of the compounds (I) also include salts of conventional bases such as, by way of example and preferably, alkali metal salts (e.g., sodium and potassium salts), alkaline earth metal salts (e.g., calcium and magnesium salts) and ammonium salts derived from ammonia or organic amines having 1 to 16 carbon atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

Solvates for the purposes of the invention refer to those forms of the compounds which form a complex in the solid or liquid state through coordination with solvent molecules. Hydrates are a special form of solvates in which coordination takes place with water.

Halogen stands for fluorine, chlorine, bromine and iodine.

A symbol # on a carbon atom means that the compound is in enantiopure form with respect to the configuration at this carbon atom, meaning in the context of the present invention an enantiomeric excess of more than 90% (>90% ee).

In the formulae of the groups which R³ can represent, the end point of the line beside which there is in each case an * does not represent a carbon atom or a CH₂ group but forms part of the bond to the nitrogen atom to which R³ is bonded.

In the formulae of the groups which R⁷ can represent, the end point of the line beside which there is in each case an * does not represent a carbon atom or a CH₂ group but forms part of the bond to the carbon atom to which R⁷ is bonded.

Preference is also given in the context of the present invention to compounds of formula

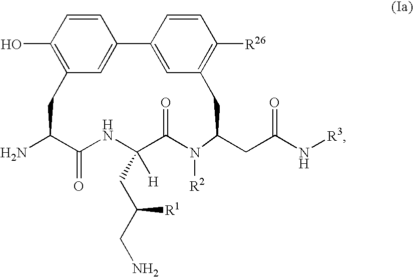

in which

R²⁶ represents hydrogen, halogen, hydroxy or methyl,

R¹ represents hydrogen or hydroxy,

R² represents hydrogen or methyl,

R³ is as defined above,

and their salts, their solvates and the solvates of their salts.

Preference is also given in the context of the present invention to compounds of formula (I) or (Ia) in which R²⁶ represents hydrogen, chlorine, hydroxy or methyl.

Preference is also given in the context of the present invention to compounds of formula (I) or (Ia) in which R²⁶ represents hydrogen.

Preference is also given in the context of the present invention to compounds of formula (Ia) in which

R²⁶ represents hydrogen or hydroxy,

R¹ represents hydrogen or hydroxy,

R² represents hydrogen,

R³ represents a group of formula

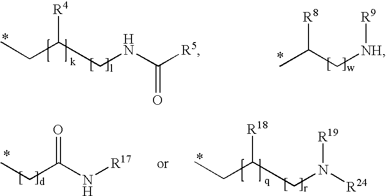

whereby

* is the linkage site to the nitrogen atom,

R⁴ represents hydrogen, amino or hydroxy,

R⁵ represents a group of formula

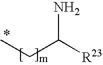

wherein

* is the linkage site to the carbon atom,

R²³ represents hydrogen or a group of formula *-(CH₂)ₙ—OH or *-(CH₂)ₒ—NH₂,

wherein

* is the linkage site to the carbon atom,
n and o independently of one another are a number 1, 2, 3 or 4,

m is a number 0 or 1,

R⁸ represents a group of formula *-CONHR¹⁴ or *-CH₂CONHR¹⁵,
wherein

* is the linkage site to the carbon atom,

R¹⁴ and R¹⁵ independently of one another represent a group of formula

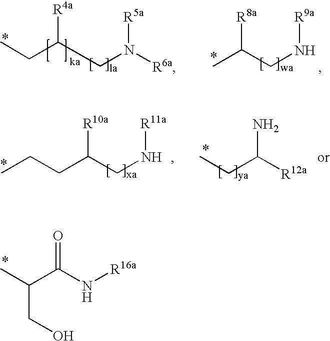

wherein

* is the linkage site to the nitrogen atom,

R^{4a} represents hydrogen, amino or hydroxy,

R^{5a} represents hydrogen, methyl or aminoethyl,

R^{6a} represents hydrogen or aminoethyl,

or

R^{5a} and R^{6a} together with the nitrogen atom to which they are bonded form a piperazine ring,

R^{8a} and R^{12a} independently of one another represent *-(CH₂)_{Z1a}—OH, *-(CH₂)_{z2a}—NHR^{3a}, *-CONHR^{14a} or *-CH₂CONHR^{5a},
wherein
   * is the linkage site to the carbon atom,

Z1a and Z2a independently of one another are a number 1, 2 or 3,

R^{13a} represents hydrogen or methyl,

and

R^{4a} and R^{15a} independently of one another represent a group of formula

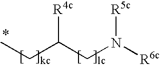

wherein

* is the linkage site to the nitrogen atom,

R^{4c} represents hydrogen, amino or hydroxy,

R^{5c} represents hydrogen, methyl or aminoethyl,

R^{6c} represents hydrogen or aminoethyl,
kc is a number 0 or 1,
and
lc is a number 1, 2, 3 or 4,

R^{9a} and R^{11a} independently of one another represent hydrogen or methyl,

R^{10a} represents amino or hydroxy,

R^{16a} represents a group of formula

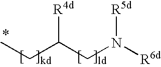

wherein

* is the linkage site to the nitrogen atom,

R^{4d} represents hydrogen, amino or hydroxy,

R^{5d} represents hydrogen, methyl or aminoethyl,

R^{6d} represents hydrogen or aminoethyl,
kd is a number 0 or 1,
and
ld is a number 1, 2, 3 or 4,

ka is a number 0 or 1,

and

la, wa, xa and ya independently of one another are a number 1, 2, 3 or 4,

R⁹ represents hydrogen, methyl, *-C(NH₂)═NH or a group of formula

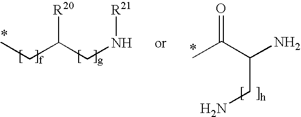

wherein

* is the linkage site to the nitrogen atom,

R²⁰ represents hydrogen or *-(CH₂)ᵢ—NHR
wherein
R²² represents hydrogen or methyl,
and
i is a number 1, 2 or 3,

R²¹ represents hydrogen or methyl,

f is a number 0, 1, 2 or 3,

g is a number 1, 2 or 3,

and

h is a number 1, 2, 3 or 4,

R¹⁷ represents a group of formula

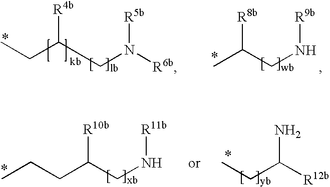

wherein

* is the linkage site to the nitrogen atom,

R^{4b} represents hydrogen, amino or hydroxy,

R^{5b} represents hydrogen, methyl or aminoethyl,

R^{6b} represents hydrogen or aminoethyl,

or

R^{5b} and R^{6b} together with the nitrogen atom to which they are bonded form a piperazine ring,

R^{8b} and R^{12b} independently of one another represent *-(CH₂)_{Z1b}—OH, *-(CH₂)_{Z2b}— NHR^{13b}, *-CONHR^{14b} or *-CH₂CONHR¹⁵
wherein
   * is the linkage site to the carbon atom,

R^{13b} represents hydrogen or methyl,

and

Z1b and Z2b independently of one another are a number 1, 2 or 3,

and

R^{4b} and R^{15b} independently of one another represent a group of formula

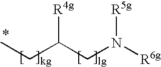

wherein

* is the linkage site to the nitrogen atom,

R^{4g} represents hydrogen, amino or hydroxy,

R^{5g} represents hydrogen, methyl or aminoethyl,

R^{6g} represents hydrogen or aminoethyl,
kg is a number 0 or 1,
and
lg is a number 1, 2, 3 or 4,

R^{9b} and R^{11b} independently of one another represent hydrogen or methyl,

R^{10b} represents amino or hydroxy,

kb is a number 0 or 1,

and

lb, wb, xb and yb independently of one another are a number 1, 2, 3 or 4,

R¹⁸ represents hydrogen, amino or hydroxy,

R¹⁹ represents hydrogen, methyl or a group of formula

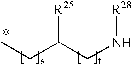

wherein

* is the linkage site to the nitrogen atom,

R²⁵ represents hydrogen or *-(CH₂)ᵤ—NHR²⁹,
wherein

R²⁹ represents hydrogen or methyl,

and

u is a number 1, 2 or 3,

R²⁸ represents hydrogen or methyl,

s is a number 0, 1, 2 or 3,

and

t is a number 1, 2 or 3,

R²⁴ represents hydrogen or aminoethyl,

d is a number 1, 2 or 3,

k and q independently of one another are a number 0 or 1,

l, r, and w independently of one another are a number 1, 2, 3 or 4,

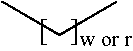

independently of one another may when w or r equals 3 carry a hydroxy group, and their salts, their solvates and the solvates of their salts.

Preference is also given in the context of the present invention to compounds of formula (Ia) in which

R²⁶ represents hydrogen or hydroxy,

R¹ represents hydrogen or hydroxy,

R² represents hydrogen,

R³ represents a group of formula

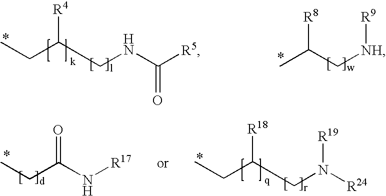

whereby

* is the linkage site to the nitrogen atom,

R⁴ represents hydrogen, amino or hydroxy,

R⁵ represents a group of formula

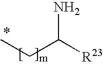

wherein

* is the linkage site to the carbon atom,

R²³ represents hydrogen or a group of formula *-(CH₂)ₙ—OH or *-(CH₂)ₒ—NH₂,

wherein

* is the linkage site to the carbon atom,
n and o independently of one another are a number 1, 2, 3, or 4,

m is a number 0 or 1,

R⁸ represents a group of formula *-CONHR¹⁴ or *-CH₂CONHR¹⁵,

wherein

* is the linkage site to the carbon atom,

R¹⁴ and R¹⁵ independently of one another represent a group of formula

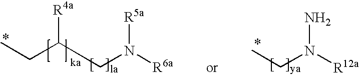

wherein

* is the linkage site to the nitrogen atom,

R^{4a} represents hydrogen, amino or hydroxy,

R^{5a} represents hydrogen,

R^{6a} represents hydrogen,

R^{12a} represents *-(CH₂)_{Z1a}—OH or *-CH₂CONHR^{15a},
wherein

* is the linkage site to the carbon atom,

Z1a is a number 1, 2 or 3,

and

R^{15a} represents a group of formula

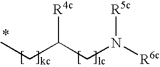

wherein

* is the linkage site to the nitrogen atom,

R^{4c} represents hydrogen, amino or hydroxy,

R^{5c} represents hydrogen,

R^{6c} represents hydrogen,
kc is a number 0 or 1,
and
lc is a number 1, 2, 3 or 4,

ka is a number 0 or 1,

and

la and ya independently of one another are a number 1, 2, 3 or 4,

R⁹ represents hydrogen,

R¹⁷ represents a group of formula

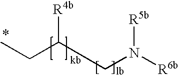

wherein

* is the linkage site to the nitrogen atom,

R^{4b} represents hydrogen, amino or hydroxy,

R^{5b} represents hydrogen,

R^{6b} represents hydrogen,

kb is a number 0 or 1,

lb is a number 1, 2, 3 or 4,

R¹⁸ represents hydrogen, amino or hydroxy,

R¹⁹ represents hydrogen,

R²⁴ represents hydrogen,

d is a number 1, 2 or 3,

k and q independently of one another are a number 0 or 1,

l, r and w independently of one another are a number 1, 2, 3 or 4,

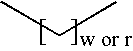

independently of one another may when w or r equals 3 carry a hydroxy group, and their salts, their solvates and the solvates of their salts.

The invention further relates to a method for preparing the compounds of formula (I) or their salts, their solvates or the solvates of their salts, whereby according to method

[A] compounds of formula

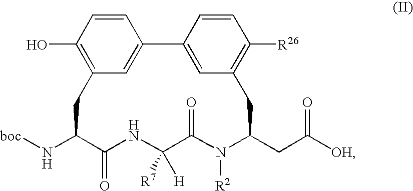

wherein R², R⁷ and R²⁶ have the meaning indicated above, and boc represents tert-butoxycarbonyl,

are reacted in a two-stage process firstly in the presence of one or more dehydrating reagents with compounds of formula

H₂NR³ (III),

wherein R³ has the meaning indicated above,

and subsequently with an acid and/or by hydrogenolysis,

or

[B] compounds of formula

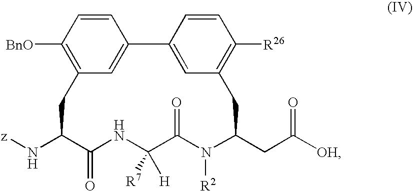

wherein R², R⁷ and R²⁶ have the meaning indicated above, and Z represents benzyloxycarbonyl,

are reacted in a two-stage process firstly in the presence of one or more dehydrating reagents with compounds of formula

H₂NR³ (III),

wherein R³ has the meaning indicated above,

and subsequently with an acid or by hydrogenolysis.

The free base of the salts can be obtained for example by chromatography on a reversed phase column using an acetonitrile-water gradient with the addition of a base, in particular by using an RP18 Phenomenex Luna C18(2) column and diethylamine as base.

The invention further relates to a method for preparing the compounds of formula (I) or their solvates according to claim 1 in which salts of the compounds or solvates of the salts of the compounds are converted into the compounds by chromatography with the addition of a base.

During the reaction with compounds of formula (III), the hydroxy group on R¹ is where appropriate protected with a tert-butyldimethylsilyl group which is removed in the second reaction step.

Reactive functionalities in the radical R³ of compounds of formula (III) are introduced into the synthesis already protected, with preference for acid-labile protecting groups (e.g., boc). After the reaction to give compounds of formula (I) has taken place, the protecting groups can be removed by deprotection reaction. This takes place by standard methods of protecting group chemistry. Deprotection reactions under acidic conditions or by hydrogenolysis are preferred.

The reaction in the first stage of methods [A] and [B] generally takes place in inert solvents, where appropriate in the presence of a base, preferably in a temperature range from 0° C. to 40° C. under atmospheric pressure.

Examples of suitable dehydrating reagents hereby are carbodiimides such as, for example, N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate, or O-(benzotriazol-1-yl)-N,N,NN′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or mixtures thereof, or mixtures thereof together with bases.

Examples of bases are alkali metal carbonates such as, for example, sodium or potassium carbonate, or sodium or potassium bicarbonate, or organic bases such as trialkylamines, e.g., triethylamine, N-methylmorpholine, N-methylpiperidine, 4-dimethylaminopyridine or diisopropylethylamine.

The condensation is preferably carried out with HATU in the presence of a base, in particular diisopropylethylamine, or with EDC and HOBt in the presence of a base, in particular triethylamine.

Examples of inert solvents are halohydrocarbons such as dichloromethane or trichloromethane, hydrocarbon such as benzene, or nitromethane, dioxane, dimethylformamide or acetonitrile. It is likewise possible to employ mixtures of the solvents. Dimethylformamide is particularly preferred.

The reaction with an acid in the second stage of methods [A] and [B] preferably takes place in a temperature range from 0° C. to 40° C. under atmospheric pressure.

Suitable acids hereby are hydrogen chloride in dioxane, hydrogen bromide in acetic acid or trifluoroacetic acid in methylene chloride.

The hydrogenolysis in the second stage of method [B] generally takes place in a solvent in the presence of hydrogen and palladium on activated carbon, preferably in a temperature range from 0° C. to 40° C. under atmospheric pressure.

Examples of solvents are alcohols such as methanol, ethanol, n-propanol or isopropanol, in a mixture with water and glacial acetic acid, with preference for a mixture of ethanol, water and glacial acetic acid.

The compounds of formula (III) are known or can be prepared in analogy to known methods.

The compounds of formula (II) are known or can be prepared by reacting compounds of formula

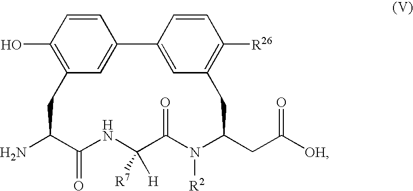

wherein R², R⁷ and R²⁶ have the meaning indicated above,

with di(tert-butyl) dicarbonate in the presence of a base.

The reaction generally takes place in a solvent, preferably in a temperature range from 0° C. to 40° C. under atmospheric pressure.

Examples of bases are alkali metal hydroxides such as sodium or potassium hydroxide, or alkali metal carbonates such as cesium carbonate, sodium or potassium carbonate, or other bases such as DBU, triethylamine or diisopropylethylamine, with preference for sodium hydroxide or sodium carbonate.

Examples of solvents are halohydrocarbons such as methylene chloride or 1,2-dichloroethane, alcohols such as methanol, ethanol or isopropanol, or water.

The reaction is preferably carried out with sodium hydroxide in water or sodium carbonate in methanol.

The compounds of formula (V) are known or can be prepared by reacting compounds of formula

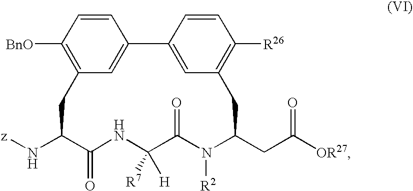

wherein R², R⁷ and R²⁶ have the meaning indicated above, and

R²⁷ represents benzyl, methyl or ethyl,

with an acid or by hydrogenolysis as described for the second stage of method [B], where appropriate by subsequent reaction with a base to hydrolyse the methyl or ethyl ester.

The hydrolysis can take place for example as described for the reaction of compounds of formula (VI) to give compounds of formula (IV).

The compounds of formula (IV) are known or can be prepared by hydrolysing the benzyl, methyl or ethyl ester in compounds of formula (VI).

The reaction generally takes place in a solvent in the presence of a base, preferably in a temperature range from 0° C. to 40° C. under atmospheric pressure.

Examples of bases are alkali metal hydroxides such as lithium, sodium or potassium hydroxide, with preference for lithium hydroxide.

Examples of solvents are halohydrocarbons such as dichloromethane or trichloromethane, ethers, such as tetrahydrofuran or dioxane, or alcohols such as methanol, ethanol or isopropanol, or dimethylformamide. It is likewise possible to employ mixtures of the solvents or mixtures of the solvents with water. Tetrahydrofuran or a mixture of methanol and water are particularly preferred.

The compounds of formula (VI) are known or can be prepared by reacting compounds of formula

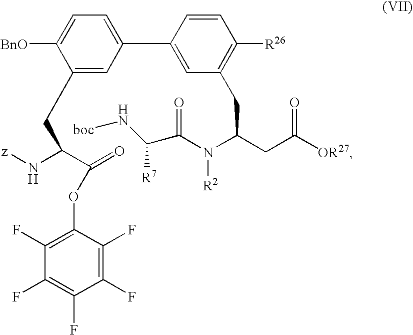

wherein R², R⁷, R²⁶ and R²⁷ have the meaning indicated above,

in the first stage with acids as described for the second stage of methods [A] and [B], and in the second stage with bases.

In the second stage the reaction with bases generally takes place in a solvent, preferably in a temperature range from 0° C. to 40° C. under atmospheric pressure.

Examples of bases are alkali metal hydroxides such as sodium or potassium hydroxide, or alkali metal carbonates such as cesium carbonate, sodium or potassium carbonate, or other bases such as DBU, triethylamine or diisopropylethylamine, with preference for triethylamine.

Examples of solvents are halohydrocarbons such as chloroform, methylene chloride or 1,2-dichloroethane, or tetrahydrofuran, or mixtures of the solvents, with preference for methylene chloride or tetrahydrofuran.

The compounds of formula (VII) are known or can be prepared by reacting compounds of formula

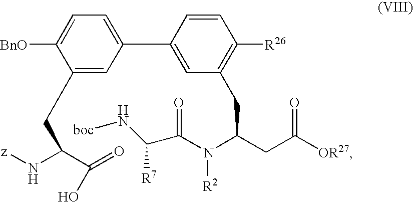

wherein R², R⁷, R²⁶ and R²⁷ have the meaning indicated above,

with pentafluorophenol in the presence of dehydrating reagents as described for the first stage of methods [A] and [B].

The reaction preferably takes place with DMAP and EDC in dichloromethane in a temperature range from −40° C. to 40° C. under atmospheric pressure.

The compounds of formula (VIII) are known or can be prepared by reacting compounds of formula

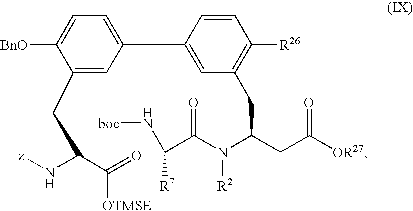

wherein R², R⁷, R²⁶ and R²⁷ have the meaning indicated above,

with fluoride, in particular with tetrabutylammonium fluoride.

The reaction generally takes place in a solvent, preferably in a temperature range from −10° C. to 30° C. under atmospheric pressure.

Examples of inert solvents are halohydrocarbons such as dichloromethane, or hydrocarbons such as benzene or toluene, or ethers such as tetrahydrofuran or dioxane, or dimethylformamide. It is likewise possible to employ mixtures of the solvents. Tetrahydrofuran and dimethylformamide are preferred solvents.

The compounds of formula (IX) are known or can be prepared by reacting compounds of formula

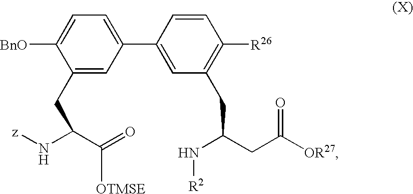

wherein R², R²⁶ and R²⁷ have the meaning indicated above,

with compounds of formula

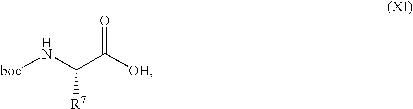

wherein R⁷ has the meaning indicated above, in the presence of dehydrating reagents as described for the first stage of methods [A] and [B].

The compounds of formula (X) are known or can be prepared in analogy to the methods described in the examples section.

The compounds of formula (XI) are known or can be prepared in analogy to known methods.

The compounds of the invention show a valuable range of pharmacological and pharmacokinetic effects which could not have been predicted.

They are therefore suitable for use as medicaments for the treatment and/or prophylaxis of diseases in humans and animals.

Due to their pharmacological properties, the compounds of the invention can, be used alone or in combination with other active ingredients for the treatment and/or prophylaxis of infectious diseases, especially of bacterial infections.

For example, it is possible to treat and/or prevent local and/or systemic diseases caused by the following pathogens or by mixtures of the following pathogens:

Gram-positive cocci, e.g., staphylococci (Staph. aureus, Staph. epidermidis) and streptococci (Strept. agalactiae, Strept. faecalis, Strept. pneumoniae, Strept. pyogenes); gram-negative cocci (neisseria gonorrhoeae) as well as gram-negative rods such as enterobacteriaceae, e.g., Escherichia coli, Haemophilus influenzae, Citrobacter (Citrob. freundii, Citrob. divernis), Salmonella and Shigella; furthermore klebsiellas (Klebs. pneumoniae, Klebs. oxytocy), Enterobacter (Ent. aerogenes, Ent. agglomerans), Hafnia, Serratia (Serr. marcescens), Proteus (Pr. mirabilis, Pr. rettgeri, Pr. vulgaris), Providencia, Yersinia, as well as the genus Acinetobacter. The antibacterial spectrum additionally includes the genus Pseudomonas (Ps. aeruginosa, Ps. maltophilia) as well as strictly anaerobic bacteria such as for example Bacteroides fragilis, representatives of the genus Peptococcus, Peptostreptococcus, as well as the genus Clostridium; furthermore mycoplasmas (M. pneumoniae, M. hominis, M. urealyticum) as well as mycobacteria, e.g., Mycobacterium tuberculosis.

The above list of pathogens is merely by way of example and is by no means to be interpreted restrictively. Examples which may be mentioned of diseases which are caused by the pathogens mentioned or mixed infections and can be prevented, improved or healed by the topically applicable preparations of the invention are:

infectious diseases in humans such as, for example, septic infections, bone and joint infections, skin infections, postoperative wound infections, abscesses, phlegmon, wound infections, infected burns, burn wounds, infections in the oral region, infections after dental operations, septic arthritis, mastitis, tonsillitis, genital infections and eye infections.

Apart from humans, bacterial infections can also be treated in other species. Examples which may be mentioned are:

Pigs: coli diarrhea, enterotoxemia, sepsis, dysentery, salmonellosis, metritis-mastitis-agalactiae syndrome, mastitis;

Ruminants (cattle, sheep, goats): diarrhea, sepsis, bronchopneumonia, salmonellosis, pasteurellosis, mycoplasmosis, genital infections;

Horses: bronchopneumonias, joint ill, puerperal and postpuerperal infections, salmonellosis;

Dogs and cats: bronchopneumonia, diarrhea, dermatitis, otitis, urinary tract infections,

prostatitis;

Poultry (chicken, turkeys, quail, pigeons, ornamental birds and others): mycoplasmosis, E. coli infections, chronic airway diseases, salmonellosis, pasteurellosis, psittacosis.

It is likewise possible to treat bacterial diseases in the rearing and management of productive and ornamental fish, whereby the antibacterial spectrum is extended beyond the pathogens mentioned above to further pathogens such as, for example, Pasteurella, Brucella, Campylobacter, Listeria, Erysipelothris, corynebacteria, Borellia, Treponema, Nocardia, Rikettsie, Yersinia.

The present invention further relates to the use of the compounds of the invention for the treatment and/or prophylaxis of diseases, preferably of bacterial diseases, especially of bacterial infections.

The present invention further relates to the use of the compounds of the invention for the treatment and/or prophylaxis of diseases, especially of the aforementioned diseases.

The present invention further relates to the use of the compounds of the invention for the production of a medicament for the treatment and/or prophylaxis of diseases, especially of the aforementioned diseases.

The present invention further relates to a method for the treatment and/or prophylaxis of diseases, especially of the aforementioned diseases, using an antibacterially effective amount of the compounds of the invention.

The compounds of the invention may act systemically and/or locally. For this purpose, they can be administered in a suitable way such as, for example, orally, parenterally, pulmonarily, nasally, sublingually, lingually, buccally, rectally, dermally, transdermally, conjuctivally, otically or as an implant or stent.

For these administration routes the compounds of the invention can be administered in suitable administration forms.

Suitable for oral administration are administration forms which function according to the prior art and deliver the compounds of the invention rapidly and/or in modified fashion, and which contain the compounds of the invention in crystalline and/or amorphicized and/or dissolved form, such as, for example, tablets (uncoated or coated tablets, for example having coatings which are resistant to gastric juice or dissolve with a delay or are insoluble and control the release of the compound of the invention), tablets or films/wafers, which disintegrate rapidly in the oral cavity, films/lyophilisates, capsules (for example hard or soft gelatin capsules), sugar-coated tablets, granules, pellets, powders, emulsions, suspensions, aerosols or solutions.

Parenteral administration can take place with avoidance of an absorption step (e.g., intravenously, intraarterially, intracardially, intraspinally or intralumbarly) or with inclusion of an absorption (e.g., intramuscularly, subcutaneously, intracutaneously, percutaneously or intraperitoneally). Administration forms suitable for parenteral administration are, inter alia, preparations for injection and infusion in the form of solutions, suspensions, emulsions, lyophilisates or sterile powders.

Suitable for the other administration routes are, for example, pharmaceutical forms for inhalation (inter alia powder inhalers, nebulizers), nasal drops, solutions, sprays; tablets, films/wafers or capsules for lingual, sublingual or buccal administration, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

The compounds of the invention can be converted into the stated administration forms. This can take place in a manner known per se by mixing with inert, nontoxic, pharmaceutically acceptable excipients. These excipients include, inter alia, carriers (for example microcrystalline cellulose, lactose, mannitol), solvents (e.g., liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g., antioxidants such as, for example, ascorbic acid), colors (e.g., inorganic pigments such as, for example, iron oxides) and taste- and/or odor-corrigents.

The present invention further relates to medicaments which comprise at least one compound of the invention, usually together with one or more inert, nontoxic, pharmaceutically acceptable excipients, and to their use for the aforementioned purposes.

It has generally proved advantageous on parenteral administration to administer amounts of about 5 to 250 mg/kg of body weight per 24 h to achieve effective results. The amount on oral administration is about 5 to 100 mg/kg of body weight per 24 h.

It may nevertheless be necessary where appropriate to deviate from the stated amounts, in particular as a function of body weight, administration route, individual behavior towards the active compound, nature of the preparation and time or interval over which administration takes place. Thus, it may be sufficient in some cases to make do with less than the aforementioned minimum amount, whereas in other cases the stated upper limit must be exceeded. In the case of an administration of larger amounts, it may be advisable to divide these into a plurality of individual doses over the day.

The percentage data in the following tests and examples are percentages by weight unless otherwise indicated; parts are parts by weight. Solvent ratios, dilution ratios and concentration data for liquid/liquid solutions are in each case based on volume.

### DETAILED DESCRIPTION OF PREFERRED EMBODIMENTS

### Examples

### Abbreviations Used:

abs. absolute
aq. aqueous
Bn benzyl
boc tert-butoxycarbonyl
CDCl₃ chloroform
CH cyclohexane
d doublet (in ¹H NMR)
dd doublet of doublets (in ¹H NMR)
DCC dicyclohexylcarbodiimide
DIC diisopropylcarbodiimide
DIEA diisopropylethylamine (Hünig's base)
DMSO dimethyl sulfoxide

### DMAP 4-N,N-dimethylaminopyridine

DMF dimethylformamide
EA ethyl acetate
EDC N′-(3-dimethylaminopropyl)-N-ethylcarbodiimide×HCl
ESI electrospray ionization (in MS)
Ex. example
Fmoc 9-fluorenylmethoxycarbonyl
HATU O-(7-azabenzotriazol-1-yl)-N,N,N′,N′-tetramethyluronium hexafluorophosphate
HBTU O-(benzotriazol-1-yl)-N,N,N′,N′-tetramethyluronium hexafluorophosphate
HOBt 1-hydroxy-1H-benzotriazole×H₂O
h hour(s)
HPLC high pressure, high performance liquid chromatography
LC-MS coupled liquid chromatography-mass spectroscopy
m multiplet (in ¹H NMR)
min minute
MS mass spectroscopy
NMR nuclear magnetic resonance spectroscopy
MTBE methyl tert-butyl ether
Pd/C palladium/carbon
PFP pentafluorophenol
PyBOP benzotriazol-1-yloxytris(pyrrolidino)phosphonium hexafluorophosphate
q quartet (in ¹H NMR)
R_{f} retention index (in TLC)
RP reverse phase (in HPLC)
RT room temperature
Rₜ retention time (in HPLC)
singlet (in ¹H NMR)
sat saturated
t triplet (in ¹H NMR)
TBS tert-butyldimethylsilyl
TFA trifluoroacetic acid
THF tetrahydrofuran
TLC thin-layer chromatography
TMSE 2-(trimethylsilyl)ethyl
TPTU 2-(2-oxo-1(2H)-pyridyl)-1,1,3,3,-tetramethyluronium tetrafluoroborate
Z benzyloxycarbonyl

### LC-MS and HPLC Methods:

Method 1 (LC-MS): Instrument: Micromass Quattro LCZ with HPLC Agilent series 1100; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

Method 2 (LC-MS): MS instrument type: Micromass ZQ; HPLC instrument type: Waters Alliance 2795; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 3 (LC-MS): MS instrument type: Micromass ZQ; HPLC instrument type: HP 1100 series; UV DAD; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 4 (LC-MS): Instrument: Micromass Platform LCZ with HPLC Agilent series 1100; column: Thermo Hypersil GOLD 3μ 20 mm×4 mm; eluent A: 1 l of water+0.5 ml of 50% formic acid, eluent B: 1 l of acetonitrile+0.5 ml of 50% formic acid; gradient: 0.0 min 100% A→0.2 min 100% A→2.9 min 30% A→3.1 min 10% A→5.5 min 10% A; flow rate: 0.8 ml/min; oven: 50° C.; UV detection: 210 nm.

### Starting Compounds

### Example 1A

### 4-(3-Bromophenyl)-3-[(tert-butoxycarbonyl)amino]butanoic acid

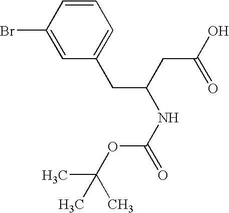

62 ml of a 1N sodium hydroxide solution are added to a solution of 8.0 g (31.1 mmol) of 3-amino-4-(3-bromophenyl)butanoic acid in 100 ml of water. While stirring, a solution of 20 g (93 mmol) of di-tert-butyl dicarbonate in 100 ml of methanol is added thereto at RT, and the mixture is stirred for 2 h. The pH is adjusted to 3 by adding 0.1N hydrochloric acid, and the mixture is extracted twice with ethyl acetate. The organic phases are combined, dried with magnesium sulfate and evaporated to dryness in vacuo. The remaining solid is used without further purification.

Yield: 7.0 g (56% of theory)

LC-MS (Method 3): Rₜ=2.55 min

MS (EI): m/z=359 (M+H)⁺

### Example 2A

### Methyl 4-(3-bromophenyl)-3-[(tert-butoxycarbonyl)amino]butanoate

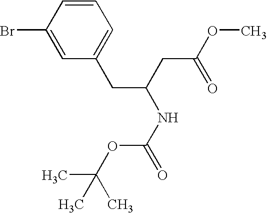

5.68 g (30 mmol) of EDC and 0.71 g (5.23 mmol) of HOBt are added to a solution, cooled to 0° C., of 7.1 g (17.4 mmol) of 4-(3-bromophenyl)-3-[(tert-butoxycarbonyl)amino]butanoic acid (Example 1A) in 100 ml of methanol. The mixture is slowly warmed to RT and stirred at RT for 12 h. The solution is concentrated in vacuo, and the residue is taken up in ethyl acetate. The organic phase is washed successively with saturated sodium bicarbonate and sodium chloride solutions, dried over magnesium sulfate and evaporated in vacuo. The remaining solid is stirred with acetonitrile, collected by filtration and dried under high vacuum.

Yield: 4.0 g (60% of theory)

LC-MS (Method 3): Rₜ=2.70 min.

MS (EI): m/z=373 (M+H)⁺.

### Example 3A

### Methyl 4-(4′-(benzyloxy)-3′-{(2S)-2-{[(benzyloxy)carbonyl]amino}-3-oxo-3-[2-(trimethylsilyl)ethoxy]propyl}biphenyl-3-yl)-3-[(tert-butoxycarbonyl)amino]butanoate

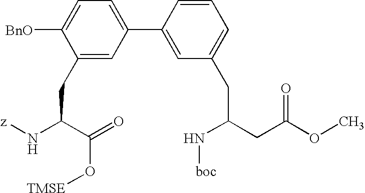

A solution of 1.0 g (2.66 mmol) of methyl 4-(3-bromophenyl)-3-[(tert-butoxycarbonyl)amino]butanoate (Example 2A) and 2.51 g (3.0 mmol) of 2-(trimethylsilyl)ethyl-2-(benzyloxy)-N-[(benzyloxy)carbonyl]-5-(4,4,5,5-tetramethyl-1,3,2-dioxaborolan-2-yl)-L-phenylalaninate (Example 84A of WO 03/106480) in 13 ml of 1-methyl-2-pyrrolidone and 1 ml of water is rendered inert and saturated with argon. Subsequently, 0.2 g (0.27 mmol) of bis(diphenylphosphino)ferrocenepalladium(II) chloride (PdCl₂ (dppf)) and 1.7 g (5.3 mmol) of cesium carbonate are added. A gentle stream of argon is passed over the reaction mixture, which is stirred at 50° C. for 6 h. The mixture is cooled, taken up in ethyl acetate and washed several times with water. The organic phase is dried over magnesium sulfate, and the solvent is concentrated in vacuo. The residue is purified by column chromatography on silica gel (cyclohexane:ethyl acetate 20:1→10:1).

Yield: 1.84 g (83% of theory).

LC-MS (Method 3): Rₜ=3.72 min

MS (EI): m/z=797 (M+H)⁺.

### Example 4A

### Methyl 3-amino-4-(4′-(benzyloxy)-3′-{(2S)-2-{[(benzyloxy)carbonyl]amino}-3-oxo-3-[2-(trimethylsilyl)ethoxy]propyl}biphenyl-3-yl)butanoate hydrochloride

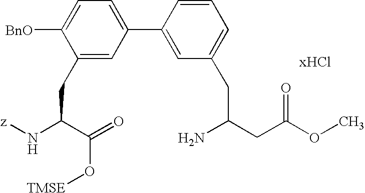

20 ml of a 4M hydrogen chloride solution in dioxane are added dropwise to a solution, cooled to 0° C., of 1.84 g (2.22 mmol) of the compound from Example 3A in 20 ml of anhydrous dioxane. After stirring at room temperature for 3 h, the solvent is evaporated in vacuo, coevaporated with dichloromethane several times and dried to constant weight under high vacuum. The crude product is reacted without further purification.

Yield: quant.

LC-MS (Method 3): Rₜ=2.38 min.

MS (EI): m/z=733 (M−HCl+H)⁺.

### Example 5A

### Methyl 4-(4′-(benzyloxy)-3′-{(2S)-2-{[(benzyloxy)carbonyl]amino}-3-oxo-3-[2-(trimethylsilyl)ethoxy]propyl}biphenyl-3-yl)-3-({(2S)-5-{[(benzyloxy)carbonyl]amino}-2-[(tert-butoxycarbonyl)amino]pentanoyl}amino)butanoate

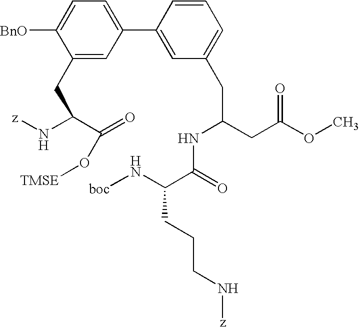

0.93 g (2.44 mmol) of HATU and 1.0 ml (6.2 mmol) of Hünig's base are added to a solution of 1.63 g (2.22 mmol) of the compound from example 4A and 0.90 g (2.44 mmol) of N⁵-[(benzyloxy)carbonyl]-N²-(tert-butoxycarbonyl)-L-ornithine in 20 ml of abs. DMF at 0° C. (bath temperature). After stirring at this temperature for 30 min, a further 0.3 ml (1.5 mmol) of Hüinig's base are added, and the temperature is allowed to rise to RT. After reaction overnight, everything is concentrated to dryness in vacuo, and the residue is taken up in dichloromethane. The organic phase is washed with water and a saturated sodium chloride solution, dried over magnesium sulfate and concentrated. The crude product is purified by chromatography on silica gel (eluent: dichloromethane/ethyl acetate 20:1→5:1).

Yield: 1.80 g (78% of theory)

LC-MS (Method 1): Rₜ=3.42 min.

MS (EI): m/z=1045 (M+H)⁺

### Example 6A

### (2S)-3-[4-(Benzyloxy)-3′-(2-{[(2S)-5-{[(benzyloxy)carbonyl]amino}-2-(carboxyamino)pentanoyl]amino}-4-methoxy-4-oxobutyl)biphenyl-3-yl]-2-{[(benzyloxy)carbonyl]amino}propanoic acid

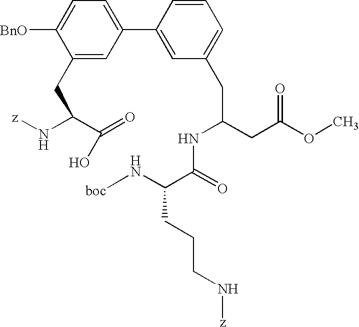

3.44 ml of a 1N tetra-n-butylammonium fluoride solution in THF are added dropwise to a solution of 1.80 g (1.72 mmol) of the compound from Example 5A in 30 ml of absolute DMF. After 1 h at RT, the mixture is cooled to 0° C., and ice-water and a few drops of 1N hydrochloric acid are added. The mixture is immediately extracted with ethyl acetate. The organic phase is dried over magnesium sulfate, concentrated in vacuo and dried under high vacuum. The crude product is reacted without further purification.

Yield: quant.

LC-MS (Method 3): Rₜ=3.06 min.

MS (EI): m/z=945 (M+H)⁺.

### Example 7A

### Methyl 4-{4′-(benzyloxy)-3′-[(2S)-2-{[(benzyloxy)carbonyl]amino}-3-oxo-3-(pentafluorophenoxy)propyl]biphenyl-3-yl}-3-({(2S)-5-{[(benzyloxy)carbonyl]amino}-2-[(tert-butoxycarbonyl)amino]pentanoyl}amino)butanoate

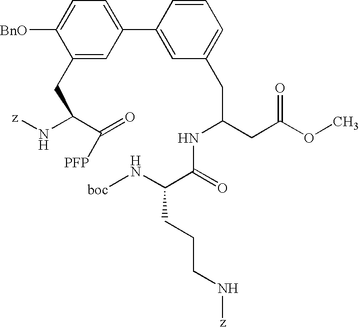

A solution of 1.63 g (1.72 mmol) of the compound from Example 6A in 55 ml of abs. dichloromethane is cooled to −25° C. and, while stirring, 0.95 g (5.2 mmol) of pentafluorophenyl, 0.021 g (0.17 mmol) of DMAP and 0.43 g (2.24 mmol) of EDC are added. The temperature is allowed to rise slowly to RT, and the mixture is stirred overnight. The mixture is concentrated in vacuo, and the crude product is dried to constant weight under high vacuum.

Yield: quant.

LC-MS (Method 2): Rₜ=3.24 min.

MS (EI): m/z=1111 (M+H)⁺

### Example 8A

### Methyl 3-[((2S)-2-amino-5-{[(benzyloxy)carbonyl]amino}pentanoyl)amino]-4-{4′-(benzyloxy)-3′-[(2S-2-{[(benzyloxy)carbonyl]amino}-3-oxo-3-(pentafluorophenoxy)propyl]biphenyl-3-yl}butanoate hydrochloride

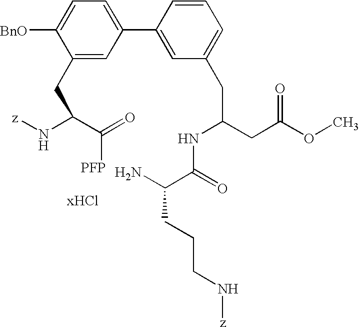

26 ml of a 4N hydrogen chloride solution in dioxane are added to a solution of 1.91 g (1.72 mmol) of the compound from Example 7A in 10 ml of dioxane while stirring at 0° C. The mixture is stirred at 0° C. for 45 min, the temperature is allowed to rise to RT, and then everything is concentrated to dryness in vacuo. The product is obtained after drying to constant weight under high vacuum.

Yield: quant.

LC-MS (Method 2): Rₜ=2.32 min.

MS (EI): m/z=1011 (M−HCl+H)⁺

### Example 9A

### Methyl[(8S11S, 14S)-17-(Benzyloxy)-14-{[(benzyloxy)carbonyl]amino}-11-(3-{[(benzyloxy)carbonyl]amino}propyl)-10,13-dioxo-9,12-diazatricyclo[14.3.1.1^{2,6}]henicosa-1(20),2(21), 3,5,16,18-hexaen-8-yl]acetate

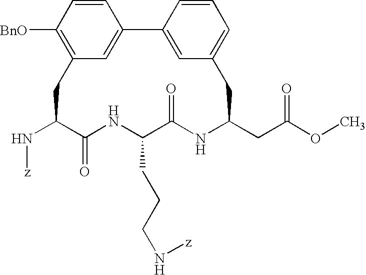

A solution of 4.8 ml (34.4 mmol) of triethylamine in 200 ml of dichloromethane is added dropwise over the course of 20 min to a solution of 1.8 g (1.72 mmol) of the compound from Example 8A in 600 ml of abs. dichloromethane with vigorous stirring. Stirring is continued overnight and everything is evaporated in vacuo (bath temperature ≦40° C.). The residue is stirred with acetonitrile, and the remaining solid is collected by filtration and dried to constant weight under high vacuum.

Yield: 0.71 g (49% of theory)

LC-MS (Method 2): Rₜ=3.02 min.

MS (EI): m/z=827 (M+H)⁺

### Example 10A

### Methyl[(8S,11S,14S)-14-amino-11-(3-aminopropyl)-17-hydroxy-10,13-dioxo-9,12-diazatricyclo[14.3.1.1^{2,6}]henicosa-1(20),2(21), 3,5,16,18-hexaen-8-yl]acetate dihydroacetate

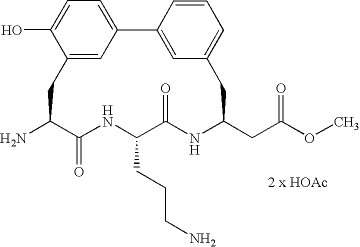

0.69 g (0.834 mmol) of the compound from Example 9A are added into a mixture of 50 ml of acetic acid/water/ethanol (4:1:1). 70 mg of palladium on activated carbon (10%) are added, and the mixture is subsequently hydrogenated under atmospheric pressure at RT for 24 h. The reaction mixture is filtered through prewashed kieselguhr, washed with ethanol, and the filtrate is concentrated on a rotary evaporator in vacuo. The residue is dried to constant weight under high vacuum.

Yield: quant.

LC-MS (Method 4): Rₜ=2.30 min.

MS (EI): m/z=469 (M−2HOAc+H)⁺.

¹H NMR (400 MHz, D₂O): δ=1.5-1.9 (m, 4H), 2.61 (m_{c}, 1H), 2.78 (m_{c}, 1H), 2.85-3.2 (m, 4H), 3.56 (m_{c}, 1H), 3.64 (s, 3H), 4.38-4.5 (m, 2H), 4.57 (m_{c}, 1H), 6.93 (d, 1H), 6.98 (s, 1H), 7.10 (d, 1H), 7.27 (s, 1H), 7.31 (t, 1H), 7.36-7.46 (m, 2H).

### Example 11A

### [(8S,11 S,14S)-14-[(tert-butoxycarbonyl)amino]-11-{3-[(tert-butoxycarbonyl)amino]propyl}-17-hydroxy-10,13-dioxo-9,12-diazotricyclo[14.3.1.1^{2,6}]henicosa-1(20), 2(21),3,5,16,18-hexaen-8-yl]acetic acid

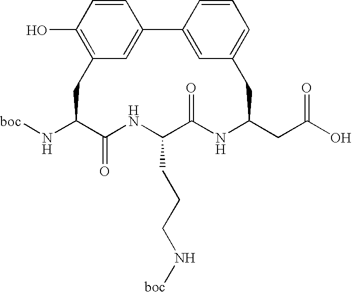

5 ml of a 1N sodium hydroxide solution are added to a solution of 0.58 g (0.99 mmol) of the compound from Example 10A in 10 ml of water. While stirring, a solution of 0.65 g (2.96 mmol) of di-tert-butyl dicarbonate in 3.7 ml of methanol is added at RT, and the mixture is stirred for 2 h. The mixture is added onto 25 ml of water, the pH is adjusted to 3 using 0.1N hydrochloric acid, and the mixture is extracted three times with ethyl acetate. The organic phases are combined, dried with magnesium sulfate and evaporated to dryness in vacuo. The remaining solid is purified to constant weight under high vacuum.

Yield: 0.46 g (71% of theory)

LC-MS (Method 1): Rₜ=2.15 min.

MS (EI): m/z=655 (M+H)⁺

### Example 12A

### Benzyl{(1S)-4-[(tert-butoxycarbonyl)amino]-1-[({2-[(tert-butoxycarbonyl)amino]ethyl}amino)carbonyl]butyl}carbamate

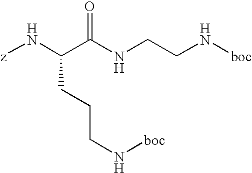

Under argon, 300 mg (0.82 mmol) of N²-[(benzyloxy)carbonyl]-N⁵-(tert-butoxycarbonyl)-L-ornithine and 171 mg (1.06 mmol) of tert-butyl-(2-aminoethyl)carbamate are dissolved in 6 ml of dimethylformamide. Then, at 0° C. (ice bath), 204 mg (1.06 mmol) of EDC and 33 mg (0.25 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and evaporated in vacuo. The remaining solid is dried under high vacuum.

Yield: 392 mg (94% of theory)

LC-MS (Method 2): Rₜ=2.36 min.

MS (ESI): m/z=509 (M+H)⁺

### Example 13A

### N⁵-(tert-Butoxycarbonyl)-N-{2-[(tert-butoxycarbonyl)amino]ethyl}-L-ornithinamide

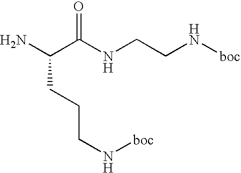

A solution of 390 mg (0.77 mmol) of benzyl {(1S)-4-[(tert-butoxycarbonyl)amino]-1-[({2-[(tert-butoxycarbonyl)amino]ethyl}amino)carbonyl]butyl}carbamate (Example 12A) in 50 ml of ethanol is hydrogenated after the addition of 40 mg of palladium on activated carbon (10%) at RT under atmospheric pressure for 4 h. The mixture is filtered through kieselguhr, and the residue is washed with ethanol. The filtrate is concentrated to dryness in vacuo. The product is reacted without further purification.

Yield: 263 mg (91% of theory)

MS (ESI): m/z=375 (M+H)⁺; 397 (M+Na)⁺.

### Example 14A

### Benzyl tert-butyl[(2S)-3-({(2S)-2,5-bis[(tert-butoxycarbonyl)amino]pentyl}amino)-3-oxopropane-1,2-diyl]biscarbamate

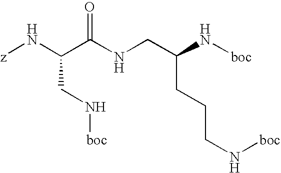

Under argon, 0.127 g (0.37 mmol) of N-[(benzyloxy)carbonyl]-3-[(tert-butoxycarbonyl)amino]-L-alanine and 0.193 g (0.49 mmol) of tert-butyl {(4S)-5-amino-4-[(tert-butoxycarbonyl)amino]pentyl}carbamate (Example 140A from WO 05/033129) are dissolved in 6 ml of dimethylformamide. Then, at 0° C. (ice bath), 0.093 g (0.49 mmol) of EDC and 0.015 g (0.11 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and evaporated in vacuo. The remaining solid is purified by preparative HPLC (Kromasil, eluent acetonitrile/0.25% aqueous trifluoroacetic acid 5:95→95:5).

Yield: 0.126 g (53% of theory)

LC-MS (Method 1): Rₜ=2.65 min.

MS (ESI): m/z=638 (M+H)⁺

### Example 15A

### tert-Butyl[(2S)-2-amino-3-({(2S)-2,5-bis[(tert-butoxycarbonyl)amino]pentyl}amino)-3-oxopropyl]carbamate

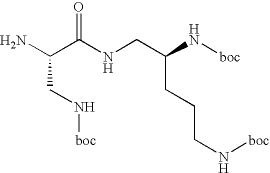

20 mg of palladium on activated carbon (10%) are added to a mixture of 0.122 g (0.19 mmol) of the compound from Example 14A in 50 ml of ethanol, and the mixture is subsequently hydrogenated under atmospheric pressure for 4 h. The reaction mixture is filtered through kieselguhr, and the filtrate is concentrated in vacuo and dried under high vacuum. The crude product is reacted without further purification.

Yield: quant.

MS (ESI): m/z=504 (M+H)⁺

### Example 16A

### Benzyl {(4S)-6-({(2S)-2,5-bis[(tert-butoxycarbonyl)amino]pentyl}amino)-4-[(tert-butoxycarbonyl)amino]-6-oxohexyl}carbamate

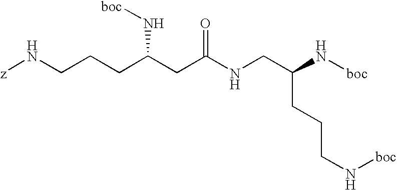

Under argon, 0.1 g (0.263 mmol) of (3S)-6-{[(benzyloxy)carbonyl]amino}-3-[(tert-butoxycarbonyl)amino]hexanecarboxylic acid (Bioorg. Med. Chem. Lett. 1998, 8, 1477-1482) and 0.108 g (0.342 mmol) of tert-butyl {(4S)-5-amino-4-[(tert-butoxycarbonyl)amino]pentyl}carbamate (Example 140A from WO 05/033129) are dissolved in 6 ml of dimethylformamide. Then, at 0° C. (ice bath), 0.066 g (0.342 mmol) of EDC and 0.011 g (0.079 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and evaporated in vacuo. The remaining solid is dried to constant weight under high vacuum.

Yield: 0.127 g (71% of theory)

LC-MS (Method 1): Rₜ=2.36 min.

MS (ESI): m/z=680 (M+H)⁺

### Example 17A

### tert-Butyl {(1S)-4-amino-1-[2-({(2S)-2,5-bis[(tert-butoxycarbonyl)amino]pentyl}amino)-2-oxoethyl]butyl}carbamate

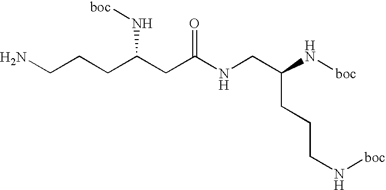

20 mg of palladium on activated carbon (10%) are added to a mixture of 0.127 g (0.19 mmol) of the compound from Example 16A in 10 ml of ethanol, and the mixture is subsequently hydrogenated under atmospheric pressure for 12 h. The reaction mixture is filtered through kieselguhr, and the filtrate is concentrated in vacuo and dried under high vacuum. The crude product is reacted without further purification.

Yield: quant.

MS (ESI): m/z=546 (M+H)⁺

### Example 18A

### Benzyl((1S,7S,12S)-7,12-bis[(tert-butoxycarbonyl)amino]-1-{3-[(tert-butoxycarbonyl)amino]propyl}-19,19-dimethyl-2,9,17-trioxo-18-oxa-3,10,16-triazaicos-1-yl)carbamate

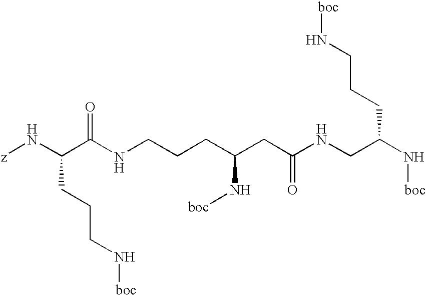

Under argon, 44 mg (0.12 mmol) of N²-[(benzyloxy)carbonyl]-N⁵-(tert-butoxycarbonyl)-L-ornithine and 85 mg (0.16 mmol) of the compound from Example 17A are dissolved in 8 ml of dimethylformamide. Then, at 0° C. (ice bath), 30 mg (0.16 mmol) of EDC and 4.9 mg (0.036 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and evaporated in vacuo. The remaining solid is dried under high vacuum.

Yield: 91 mg (85% of theory)

LC-MS (Method 1): Rₜ=2.35 min.

MS (ESI): m/z=894 (M+H)⁺

### Example 19A

### tert-Butyl {(4S,10S,15S)-4-amino-10,15-bis[(tert-butoxycarbonyl)amino]-22,22-dimethyl-5,12,20-trioxo-21-oxa-6,13,19-triazatricos-1-yl}carbamate

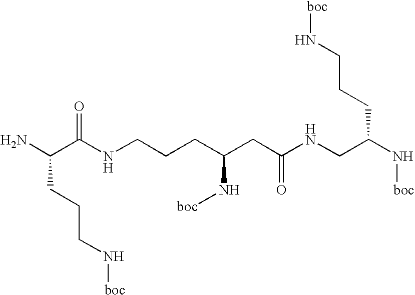

A solution of 91 mg (0.10 mmol) of the compound from Example 18A in 10 ml of ethanol is hydrogenated after the addition of 10 mg of palladium on activated carbon (10%) at RT under atmospheric pressure for 12 h. The mixture is filtered through kieselguhr and the residue is washed with ethanol. The filtrate is concentrated to dryness in vacuo. The product is reacted without further purification.

Yield: quant.

MS (ESI): m/z=760 (M+H)⁺.

### Example 20A

### Benzyl {(1S)-1-[2-({(2S)-2,5-bis[(tert-butoxycarbonyl)amino]pentyl}amino)-2-oxoethyl]-4-[(tert-butoxycarbonyl)amino]butyl}carbamate

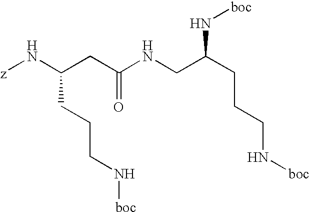

Under argon, 0.1 g (0.26 mmol) of (3S)-3-{[(benzyloxy)carbonyl]amino)-6-[(tert-butoxycarbonyl)amino]hexanecarboxylic acid (J. Med. Chem. 2002, 45, 4246-4253) and 0.11 g (0.34 mmol) of tert-butyl {(4S)-5-amino-4-[(tert-butoxycarbonyl)amino]pentyl}carbamate (Example 140A from WO 05/033129) are dissolved in 6 ml of dimethylformamide. Then, at 0° C. (ice bath), 0.065 g (0.34 mmol) of EDC and 0.011 g (0.079 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and evaporated in vacuo. The remaining solid is dried to constant weight under high vacuum.

Yield: 0.146 g (82% of theory)

LC-MS (Method 2): Rₜ=2.5 min.

MS (ESI): m/z=680 (M+H)⁺

### Example 21A

### tert-Butyl[(4S)-4-amino-6-(}(2S)-2,5-bis[(tert-butoxycarbonyl)amino]pentyl}amino)-6-oxohexyl]carbamate

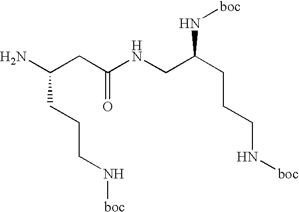

22 mg of palladium on activated carbon (10%) are added to a mixture of 0.146 g (0.22 mmol) of the compound from Example 20A in 10 ml of ethanol, and the mixture is subsequently hydrogenated under atmospheric pressure for 12 h. The reaction mixture is filtered through kieselguhr, the filtrate is concentrated in vacuo and dried under high vacuum. The crude product is reacted without further purification.

Yield: quant.

MS (ESI): m/z=546 (M+H)⁺

### Example 22A

### Benzyl {(1S)-4-[(tert-butoxycarbonyl)amino]-1-[({(4S)-4-[(tert-butoxycarbonyl)amino]-5-hydroxypentyl}amino)carbonyl]butyl}carbamate

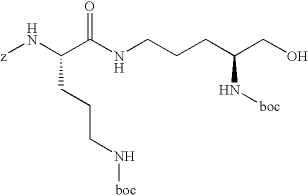

Under argon 0.155 g (0.42 mmol) of N²-[(benzyloxy)carbonyl]-N⁵-(tert-butoxycarbonyl)-L-ornithine and 0.12 g (0.55 mmol) of tert-butyl[(1S)-4-amino-1-(hydroxymethyl)butyl]carbamate (Example 172A from WO 05/033129) are dissolved in 6 ml of dimethylformamide. Then, at 0° C. (ice bath), 0.105 g (0.55 mmol) of EDC and 0.017 g (0.13 mmol) of HOBt are added. The mixture is slowly warmed to RT and stirred at RT for 12 h. The solution is concentrated in vacuo, and the residue is taken up in ethyl acetate. The organic phase is washed successively with saturated sodium bicarbonate and sodium chloride solutions, dried over magnesium sulfate and evaporated in vacuo. The remaining solid is purified by preparative HPLC (Kromasil, eluent acetonitrile/0.25% aqueous trifluoroacetic acid 5:95→95:5).

Yield: 0.164 g (69% of theory)

LC-MS (Method 2): Rₜ=2.05 min.

MS (EI): m/z=567 (M+H)⁺

### Example 23A

### N⁵-(tert-Butoxycarbonyl)-N-{(4s)-4-[(tert-butoxycarbonyl)amino]-5-hydroxypentyl}-L-ornithinamide

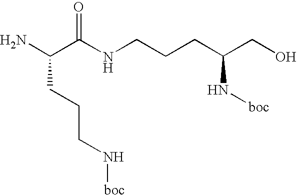

30 mg of palladium on activated carbon (10%) are added to a mixture of 0.164 g (0.29 mmol) of the compound from Example 22A in 10 ml of ethanol, and the mixture is subsequently hydrogenated under atmospheric pressure for 12 h. The reaction mixture is filtered through kieselguhr, the filtrate is concentrated in vacuo and dried under high vacuum. The crude product is reacted without further purification.

Yield: 0.125 g (quant.)

MS (EI): m/z=433 (M+H)⁺

### Example 24A

### Benzyl {(1S)-4-{[(benzyloxy)carbonyl]amino}-1-[({3-[(tert-butoxycarbonyl)amino]-2-hydroxypropyl}amino)carbonyl]butyl}carbamate

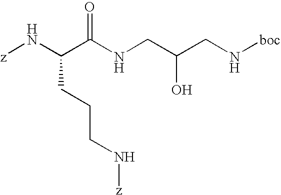

Under argon 0.20 g (0.50 mmol) of N²,N⁵-bis[(benzyloxy)carbonyl]-L-ornithine and 0.124 g (0.65 mmol) of tert-butyl-(3-amino-2-hydroxypropyl)carbamate are dissolved in 6 ml of dimethylformamide. Then, at 0° C. (ice bath), 0.124 g (0.65 mmol) of EDC and 0.02 g (0.15 mmol) of HOBt are added. The mixture is slowly warmed to RT and stirred at RT for 12 h. The solution is concentrated in vacuo, and the residue is taken up in ethyl acetate. The organic phase is washed successively with saturated sodium bicarbonate and sodium chloride solutions, dried over magnesium sulfate and evaporated in vacuo. The remaining solid is dried to constant weight under high vacuum.

Yield: 0.245 g (86% of theory)

LC-MS (Method 2): Rₜ=2.15 min.

MS (EI): m/z=573 (M+H)⁺

### Example 25A

### Benzyl((4S)-5-[(3-amino-2-hydroxypropyl)amino]-4-{[(benzyloxy)carbonyl]amino}-5-oxopentyl)carbamate hydrochloride

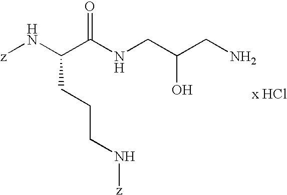

6.8 ml of a 4N hydrogen chloride solution in dioxane are added to a solution of 0.263 g (0.46 mmol) of the compound from Example 24A in 1 ml of dioxane at 0° C. After 2 h at RT, the reaction solution is concentrated in vacuo and coevaporated several times with di-chloromethane. The remaining solid is dried to constant weight under high vacuum.

Yield: 0.205 g (88% of theory)

LC-MS (Method 2): Rₜ=1.47 min.

MS (EI): m/z=473 (M−HCl+H)⁺

### Example 26A

### Benzyl[3-({(2S)-2,5-bis[(tert-butoxycarbonyl)amino]pentyl}amino)-3-oxopropyl]carbamate

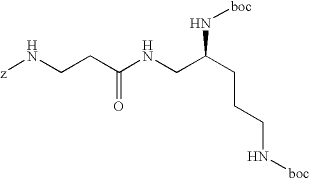

Under argon 0.10 g (0.45 mmol) of N-[(benzyloxy)carbonyl]-beta-alanine and 0.185 g (0.58 mmol) of tert-butyl {(4S)-5-amino-4-[(tert-butoxycarbonyl)amino]pentyl}carbamate (Example 140A from WO 05/033129) are dissolved in 6 ml of dimethylformamide. Then, at 0° C. (ice bath), 0.112 g (0.58 mmol) of EDC and 0.018 g (0.134 mmol) of HOBt are added. The mixture is slowly warmed to RT and stirred at RT for 12 h. The solution is concentrated in vacuo, and the residue is taken up in ethyl acetate. The organic phase is washed successively with saturated sodium bicarbonate and sodium chloride solutions, dried over magnesium sulfate and concentrated in vacuo. The remaining solid is dried to constant weight under high vacuum.

Yield: 0.215 g (92% of theory)

LC-MS (Method 2): Rₜ=2.19 min.

MS (EI): m/z=523 (M+H)⁺

### Example 27A

### tert-Butyl {(4S)-5-[(3-aminopropanoyl)amino]-4-[(tert-butoxycarbonyl)amino]pentyl}carbamate

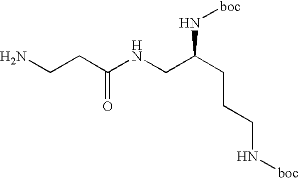

40 mg of palladium on activated carbon (10%) are added to a mixture of 0.215 g (0.41 mmol) of the compound from Example 26A in 10 ml of ethanol, and the mixture is subsequently hydrogenated under atmospheric pressure for 12 h. The reaction mixture is filtered through kieselguhr, the filtrate is concentrated in vacuo and dried under high vacuum. The crude product is reacted without further purification.

Yield: 0.160 g (quant.)

MS (EI): m/z=389 (M+H)⁺

### Example 28A

### tert-Butyl[(4S)-6-({(2S)-2,5-bis[(tert-butoxycarbonyl)amino]pentyl}amino)-4-({[(8S,11S,14S)-14-[(tert-butoxycarbonyl)amino]-11-{3-[(tert-butoxycarbonyl)amino]propyl}-17-hydroxy-10,13-dioxo-9,12-diazatricyclo[14.3.1.1^{2,6}]henicosa-1(20),2(21)3,5,16,18-hexaen-8-yl]acetyl}amino)-6-oxohexyl]carbamate

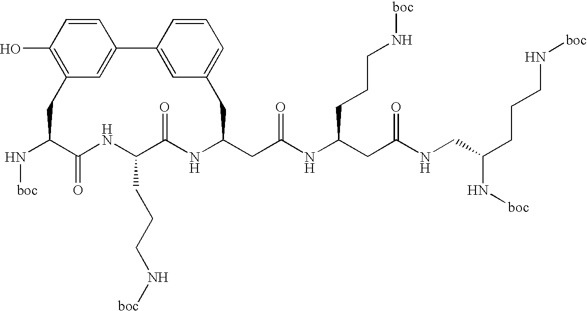

30 mg (0.046 mmol) of [(8S,11S,14S)-14-[(tert-butoxycarbonyl)amino]-11-{3-[(tert-butoxycarbonyl)amino]propyl}-17-hydroxy-10,13-dioxo-9,12-diazatricyclo[14.3.1.1^{2,6}]henicosa-1(20),2(21),3,5,16,18-hexaen-8-yl]acetic acid (Example 11A) and 32.5 mg (0.06 mmol) of the compound from Example 21A are dissolved in 2.0 ml of DMF and cooled to 0° C. 11.4 mg (0.06 mmol) of EDC and 1.9 mg (0.014 mmol) of HOBt are added, and the mixture is stirred at room temperature for 12 h. The reaction mixture is concentrated on a rotary evaporator in vacuo and purified by chromatography on Sephadex LH20 (eluent: methanol/acetic acid 0.25%).

Yield: 21 mg (38% of theory)

LC-MS (Method 3): Rₜ=2.79 min.

MS (ESI): m/z=1182 (M+H)⁺

### Example 29A

### tert-Butyl {3-[(8S,11S,14S)-8-[2-({(2S)-2,5-bis[(tert-butoxycarbonyl)amino]pentyl}amino)-2-oxoethyl]-14-[(tert-butoxycarbonyl)amino]-17-hydroxy-10,13-dioxo-9,12-diazatricyclo[14.3.1.1^{2,6}]henicosa-1(20), 2(21), 3,5,16,18-hexaen-11-yl]propyl}carbamate

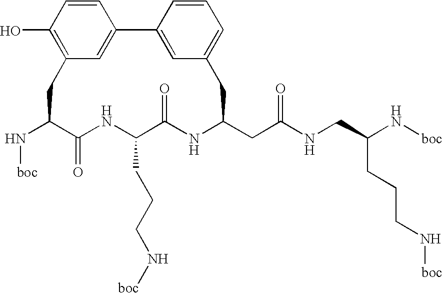

30 mg (0.046 mmol) of [(8S,11S,14S)-14-[(tert-butoxycarbonyl)amino]-11-{3-[(tert-butoxycarbonyl)amino]propyl}-17-hydroxy-10,13-dioxo-9,12-diazatricyclo[14.3.1.1^{2,6}]henicosa-1(20),2(21),3,5,16,18-hexaen-8-yl]acetic acid (Example 11A) and 18.9 mg (0.06 mmol) of tert-butyl {(4S)-5-amino-4-[(tert-butoxycarbonyl)amino]pentyl}carbamate (Example 140A from WO 05/033129) are dissolved in 2.0 ml of DMF and cooled to 0° C. 11.4 mg (0.06 mmol) of EDC and 1.9 mg (0.014 mmol) of HOBt are added, and the mixture is stirred at room temperature for 12 h. The reaction mixture is concentrated on a rotary evaporator in vacuo and purified by chromatography on Sephadex LH20 (eluent: methanol/acetic acid 0.25%).

Yield: 21 mg (48% of theory)

LC-MS (Method 1): Rₜ=2.63 min.

MS (ESI): m/z=954 (M+H)⁺

### Example 30A

### Benzyl[(1S)-4-{[(benzyloxy)carbonyl]amino}-1-({[3-({[(8S,11S,14S)-14-[(tert-butoxycarbonyl)amino]-11-{3-[(tert-butoxycarbonyl)amino]propyl}-17-hydroxy-10,13-dioxo-9,12-diazatricyclo[14.3.1.1^{2,6}]henicosa-1(20),2(21), 3,5,16,18-hexaen-8-yl]acetyl}amino)-2-hydroxypropyl]amino}carbonyl)butyl]carbamate

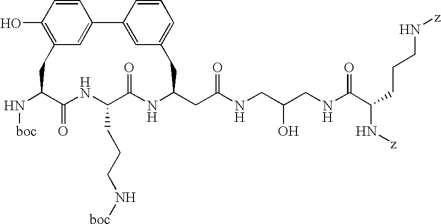

35 mg (0.053 mmol) of [(8S,11S,14S)-14-[(tert-butoxycarbonyl)amino]-11-{3-[(tert-butoxycarbonyl)amino]propyl}-17-hydroxy-10,13-dioxo-9,12-diazatricyclo[14.3.1.1^{2,6}]henicosa-1(20),2(21),3,5,16,18-hexaen-8-yl]acetic acid (Example 11A) and 35.4 mg (0.069 mmol) of the compound from Example 25A are dissolved in 2.0 ml of DMF and cooled to 0° C. 30.6 mg (0.06 mmol) of PyBOP and 0.03 ml of diisopropylethylamine are added, and the mixture is stirred at room temperature for 12 h. The reaction mixture is concentrated on a rotary evaporator in vacuo, and the residue is stirred with acetonitrile/water (1:1), collected by filtration and dried to constant weight under high vacuum.

Yield: 16 mg (27% of theory)

LC-MS (Method 3): Rₜ=2.58 min.

MS (ESI): m/z=1109 (M+H)⁺

### Example 31A

### tert-Butyl {3-[(8S,11S,14S)-14-[(tert-butoxycarbonyl)amino]-17-hydroxy-8-(2-{[2-hydroxy-3-(L-ornithylamino)propyl]amino}-2-oxoethyl)-10,13-dioxo-9,12-diazatricyclo[14.3.1.1^{2,6}]henicosa-1(20), 2(21),3,5,16,18-hexaen-11-yl]propyl}carbamate

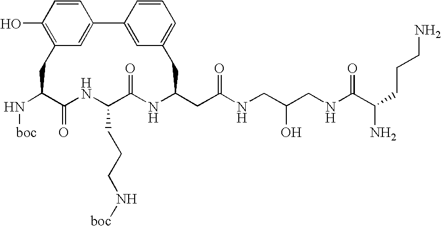

5 mg of palladium on activated carbon (10%) are added to a mixture of 15.8 mg (0.014 mmol) of the compound from Example 30A in 5 ml of ethanol, and the mixture is subsequently hydrogenated under atmospheric pressure for 12 h. The reaction mixture is filtered through a Millipore filter, the filtrate is concentrated in vacuo and dried under high vacuum. The crude product is reacted without further purification.

Yield: 11.8 mg (99% of theory)

LC-MS (Method 2): Rₜ=1.21 min.

MS (ESI): m/z=841 (M+H)⁺

### Example 32A

### tert-Butyl[(4S,10S,15S)-10,15-bis[(tert-butoxycarbonyl)amino]-4-({[(8S,11S,14S)-14-[(tert-butoxycarbonyl)amino]-11-{3-[(tert-butoxycarbonyl)amino]propyl}-17-hydroxy-10,13-dioxo-9,12-diazatricyclo[14.3.1.1^{2,6}]henicosa-1(20),2(21),3,5,16,18-hexaen-8-yl]acetyl}amino)-22,22-dimethyl-5,12,20-trioxo-21-oxa-6,13,19-triazatricos-1-yl]carbamate

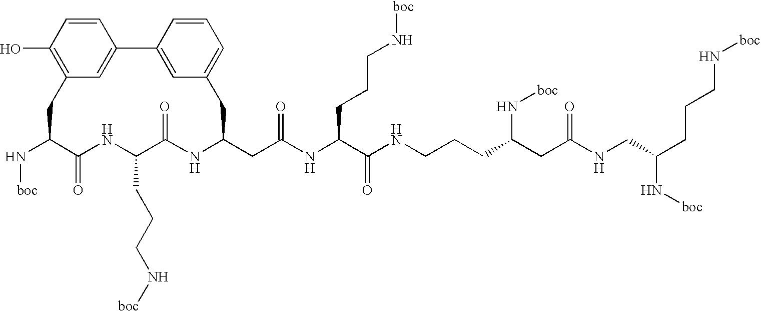

27.3 mg (0.042 mmol) of [(8S,11S,14S)-14-[(tert-butoxycarbonyl)amino]-11-{3-[(tert-butoxycarbonyl)amino]propyl}-17-hydroxy-10,13-dioxo-9,12-diazatricyclo[14.3.1.1^{2,6}]henicosa-1(20),2(21),3,5,16,18-hexaen-8-yl]acetic acid (Example 11A) and 38 mg (0.05 mmol) of the compound from Example 19A are dissolved in 2.0 ml of DMF and cooled to 0° C. 10.4 mg (0.054 mmol) of EDC and 1.7 mg (0.013 mmol) of HOBt are added, and the mixture is stirred at room temperature for 12 h. The reaction mixture is concentrated on a rotary evaporator in vacuo and purified by chromatography on Sephadex LH20 (eluent: methanol/acetic acid 0.25%).

Yield: 19.2 mg (33% of theory)

LC-MS (Method 3): Rₜ=2.87 min.

MS (ESI): m/z=1396 (M+H)⁺

Examples 33A to 37A listed in the following table are prepared in analogy to the procedure of Example 28A.

| Example | Precursor | | |
|---|---|---|---|
| No. | Example | Structure | Analytical Data |
| 33A | 11A + 13A | 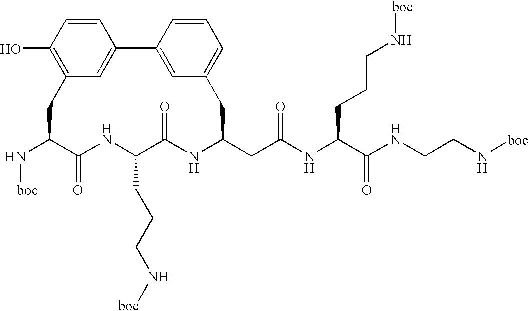 | LC-MS (Method 2):Rₜ = 2.41 min.MS (ESI): m/z =1011 (M + H)⁺. |
| 34A | 11A +tert-butyl-(2-amino-ethyl)carbamate | 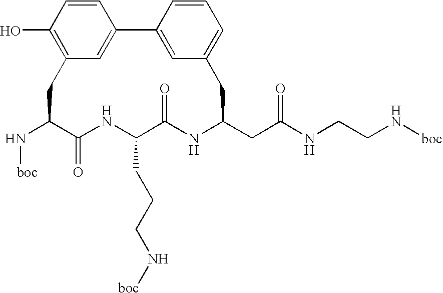 | LC-MS (Method 2):Rₜ = 2.42 min.MS (ESI): m/z = 797(M + H)⁺. |
| 35A | 11A + 23A | 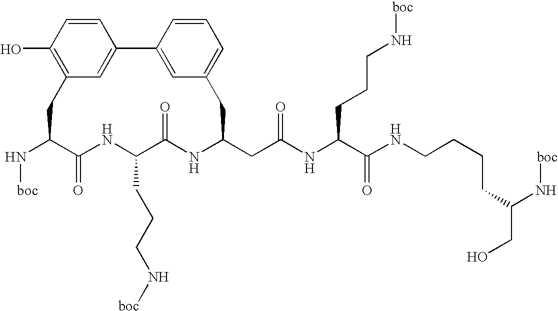 | LC-MS (Method 2):Rₜ = 2.34 min.MS (ESI): m/z =1083 (M + H)⁺. |
| 36A | 11A + 15A | 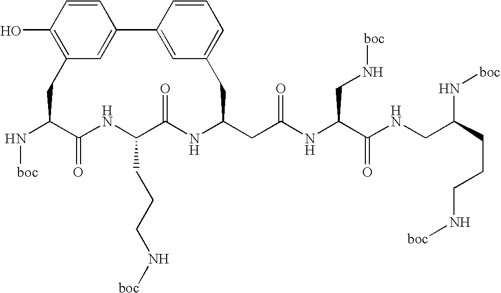 | LC-MS (Method 3):Rₜ = 3.95 min.MS (ESI): m/z =1140 (M + H)⁺. |
| 37A | 11A + 27A | 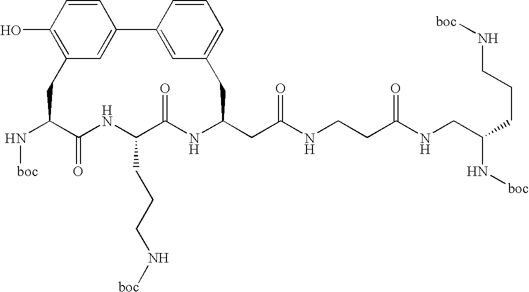 | LC-MS (Method 1):Rₜ = 2.50 min.MS (ESI): m/z =1026 (M + H)⁺. |

### EXEMPLARY EMBODIMENTS

### Example 1

### (3S)-6-Amino-3-({[(8S11,S14S)-14-amino-11-(3-aminopropyl)-17-hydroxy-10,13-dioxo-9,12-diazatricyclo[14.3.1.1^{2,6}]henicosa-1(20),2(21),3,5,16,18-hexaen-8-yl]acetyl{amino)-N-[(2S)-2,5-diaminopentyl]hexanamide pentahydrochloride

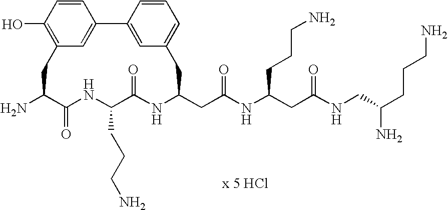

2 ml of a 4N hydrogen chloride solution in dioxane are added to a solution of 20.7 mg (0.018 mmol) of the compound from Example 28A in 1 ml of dioxane at 0° C. After 2 h at RT, the reaction solution is concentrated in vacuo and coevaporated with dichloromethane several times. The remaining solid is dried to constant weight under high vacuum.

Yield: 14 mg (93% of theory)

MS (ESI): m/z=682 (M−5HCl+H)⁺.

¹H NMR (400 MHz, D₂O): δ=1.45-1.95 (m, 12H), 2.3-2.6 (m, 3H), 2.75 (m_{c}, 1H), 2.9-3.2 (m, 1H), 3.3-3.7 (m, 3H), 4.13 (m_{c}, 1H), 4.32 (m_{c}, 1H), 4.47 (m_{c}, 1H), 6.9-7.0 (m, 2H), 7.09 (d, 1H), 7.28 (s, 1H), 7.33 (t, 1H), 7.4-7.5 (m, 2H).

The examples 2 to 9 listed below are prepared in analogy to the method of Example 1 from the corresponding starting materials.

### Example 2

### N²-{[(8S,11S,14S)-14-Amino-11-(3-aminopropyl)-17-hydroxy-10,13-dioxo-9,12-diazatricyclo[14.3.1.1^{2,6}]henicosa-1(20), 2(21),3,5,5,16,18-hexaen-8-yl]acetyl}-N-(2-aminoethyl)-L-ornithinamide tetrahydrochloride

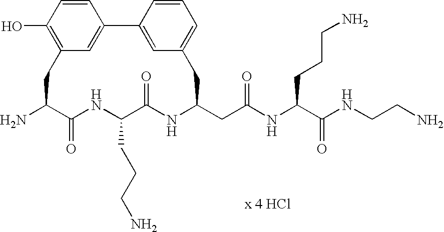

Prepared from Example 33A; yield: 93% of theory.

LC-MS (Method 3): Rₜ=0.56 min.

MS (ESI): m/z=611 (M−4HCl+H)⁺.

¹H NMR (400 MHz, D₂O): δ=1.55-1.95 (m, 8H), 2.48 (m_{c}, 1H), 2.7-2.85 (m, 2H), 2.87-3.2 (m, 7H), 3.35-3.8 (m, 4H), 4.17 (m_{c}, 1H), 4.40 (m_{c}, 1H), 4.48 (m_{c}, 1H), 4.58 (m_{c}, 1H), 6.9-7.0 (m, 2H), 7.09 (d, 1H), 7.28 (s, 1H), 7.33 (t, 1H), 7.4-7.5 (m, 2H).

### Example 3

### 2-[(8S,11S,14S)-14-Amino-11-(3-aminopropyl)-17-hydroxy-10,13-dioxo-9,12-diazatricyclo[14.3.1.1^{2,6}]henicosa-1(20), 2(21),3,5,16,18-hexaen-8-yl]-N-(2-aminoethyl)acetamide trihydrochloride

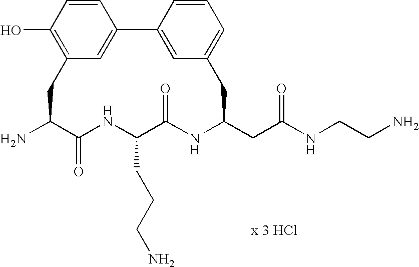

Prepared from Example 34A; yield: 81% of theory.

LC-MS (Method 4): Rₜ=1.74 min.

MS (ESI): m/z=497 (M−3HCl+H)⁺.

1H NMR (400 MHz, D₂O): δ=1.6-1.9 (m, 4H), 2.45 (m_{c}, 1H), 2.67 (m_{c}, 1H), 2.7-3.2 (m, 7H), 3.34-3.62 (m, 3H), 4.38-4.50 (m, 2H), 4.57 (m_{c}, 1H), 6.93 (d, 1H), 6.97 (s, 1H), 7.10 (d, 1H), 7.28 (s, 1H), 7.32 (t, 1H), 7.37-7.46 (m, 2H).

### Example 4

### N²-{[(8S,11S,14S)-14-Amino-11-(3-aminopropyl)-17-hydroxy-10,13-dioxo-9,12-diazatricyclo[14.3.1.1^{2,6}]henicosa-1(20),2(21), 3,5,16,18-hexaen-8-yl]acetyl}-N-[(5S)-5-amino-6-hydroxyhexyl]-L-ornithinamide tetrahydrochloride

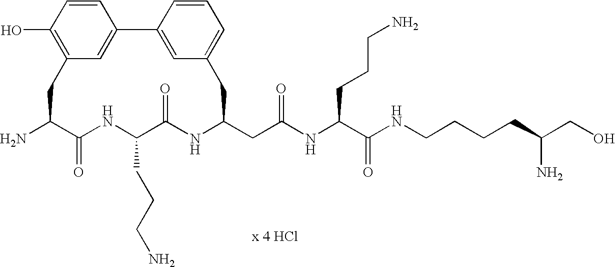

Prepared from Example 35A; yield: 97% of theory.

LC-MS (Method 2): Rₜ=0.32 min.

MS (ESI): m/z=682 (M−4HCl+H)⁺.

¹H NMR (400 MHz, D₂O): δ=1.25-1.95 (m, 14H), 2.48 (m_{c}, 1H), 2.62-2.83 (m, 2H), 2.85-3.30 (m, 8H), 3.45-3.8 (m, 4H), 4.12 (m_{c}, 1H), 4.40 (m_{c}, 1H), 4.48 (m_{c}, 1H), 4.58 (m_{c}, 1H), 6.93 (d, 1H), 6.98 (s, 1H), 7.10 (d, 1H), 7.29 (s, 1H), 7.33 (t, 1H), 7.4-7.5 (m, 2H).

### Example 5

### 2-[(8S,11S,14S)-14-Amino-11-(3-aminopropyl)-17-hydroxy-10,13-dioxo-9,12-diazatricyclo[14.3.1.1^{2,6}]henicosa-1(20),2(21),3,5,16,18-hexaen-8-yl]-N-[(2S)-2,5-diaminopentyl]acetamide tetrahydrochloride

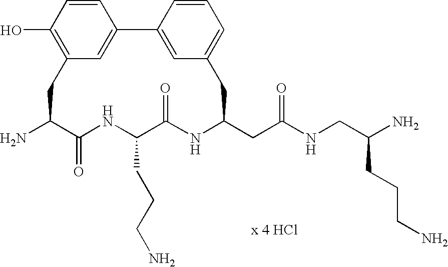

Prepared from Example 29A; yield: 97% of theory.

MS (ESI): m/z=554 (M−4HCl+H)+.

1H NMR (400 MHz, D₂O); δ=1.55-1.9 (m, 8H), 2.48 (mc, 1H), 2.67 (mc, 1H), 2.8-3.2 (m, 7H), 3.3-3.7 (m, 4H), 4.38-4.50 (m, 2H), 4.58 (mc, 1H), 6.9-7.0 (m, 2H), 6.95 (mc, 1H), 7.25-7.50 (m 4H).

### Example 6

### 3-Amino-N²-{[(8S,11S,14S)-14-amino-11-(3-aminopropyl)-17-hydroxy-10,13-dioxo-9,12-diazatricyclo[14.3.1.1^{2,6}]henicosa-1(20),2(21),3,5,16,18-hexaen-8-yl]acetyl}-N-(2,5-diaminopentyl)-L-alaninamide tetrahydrochloride

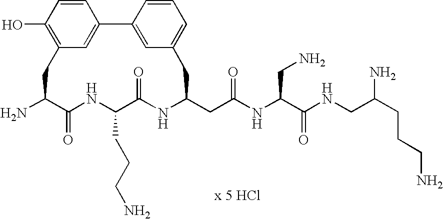

Prepared from Example 36A; yield: 99% of theory.

MS (ESI): m/z=640 (M−5HCl+H)+.

1H NMR (400 MHz, D₂O): δ=1.5-1.9 (m, 8H), 2.53 (mc, 1H), 2.68-2.85 (m, 2H), 2.9-3.1 (m, 7H), 3.23 (mc, 1H), 3.33-3.63 (m, 4H), 4.48 (m, 2H), 4.55-4.75 (m, 2H, underneath D2O), 6.91-7.0 (m, 2H), 7.10 (d, 1H), 7.29 (s, 1H), 7.34 (t, 1H), 7.4-7.5 (m, 2H).

### Example 7

### (3S)-3-Amino-6-[(N²-{[(8S,11S,14S)-14-amino-11-(3-aminopropyl)-17-hydroxy-10,13-dioxo-9,12-diazatricyclo[14.3.1.1^{2,6}]henicosa-1(20), 2(21), 3,5,16,18-hexaen-8-yl]acetyl}-L-ornithyl)amino]-N-[(2S)-2,5-diaminopentyl]hexanamide hexahydrochloride

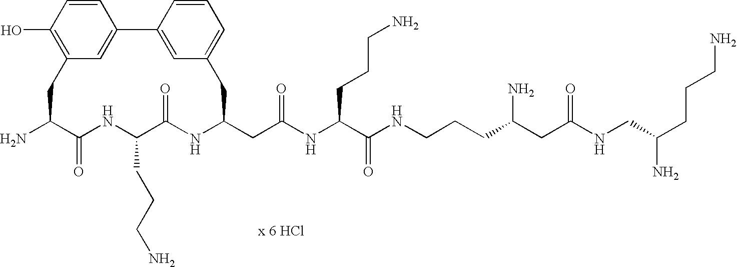

Prepared from Example 32A; yield: 99% of theory.

LC-MS (Method 3): Rₜ=0.23 min.

MS (ESI): m/z=796 (M−6HCl+H)⁺.

1H NMR (400 MHz, D₂O): δ=1.45-1.95 (m, 16H), 2.45-2.6 (m, 2H), 2.62-2.85 (m, 3H), 2.9-3.7 (m, 15H), 4.12 (m_{c}, 1H), 4.40 (m_{c}, 1H), 4.48 (m_{c}, 1H), 4.58 (m_{c}, 1H), 6.91-7.0 (d, 1H), 7.09 (s, 1H), 7.27 (d, 1H), 7.27 (s, 1H), 7.32 (t, 1H), 7.38-7.46 (m, 2H).

### Example 8

### N³-{[(8S,11S,14S)-14-Amino-11-(3-aminopropyl)-17-hydroxy-10,13-dioxo-9,12-diazatricyclo[14.3.1.1^{2,6}]henicosa-1(20),2(21), 3,5,16,18-hexaen-8-yl]acetyl}-N-[(2S)-2,5-diaminopentyl]-beta-alaninamide tetrahydrochloride

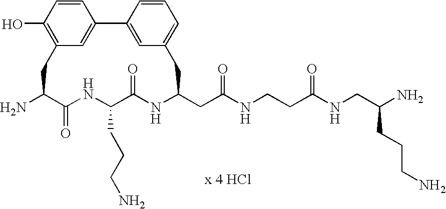

Prepared from Example 37A; yield: 99% of theory.

LC-MS (Method 3): Rₜ=0.26 min.

MS (ESI): m/z=625 (M−4HCl+H)⁺.

¹H NMR (400 MHz, D₂O): δ=1.55-1.9 (m, 8H), 2.38-2.5 (m, 3H), 2.58 (m_{c}, 1H), 2.7-3.2 (m, 8H), 3.21-3.6 (m, 5H), 4.36 (m_{c}, 1H), 4.48 (m_{c}, 1H), 4.58 (m_{c}, 1H), 6.94 (d, 1H), 6.99 (s, 1H), 7.10 (d, 1H), 7.28 (s, 1H), 7.32 (t, 1H), 7.37-7.47 (m, 2H).

### Example 9

### N-[3-({[(8S 11S, 14S)-14-Amino-11-(3-aminopropyl)-17-hydroxy-10,13-dioxo-9,12-diazatricyclo[14.3.1.1^{2,6}]henicosa-1(20), 2(21), 3,5,16,18-hexaen-8-yl]acetyl}amino)-2-hydroxypropyl]-L-ornithinamide tetrahydrochloride

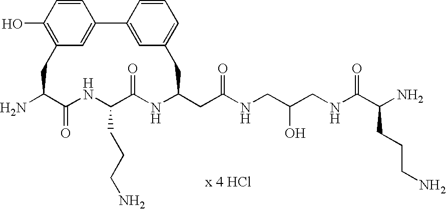

Prepared from Example 31A; yield: 73% of theory.

LC-MS (Method 2): Rₜ=0.28 min.

MS (ESI): m/z=641 (M−4HCl+H)⁺.

¹H NMR (400 MHz, D₂O): δ=1.58-1.95 (m, 8H), 2.48 (m_{c}, 1H), 2.64 (m_{c}, 1H), 2.77 (m_{c}, 1H), 2.85-3.4 (m, 9H), 3.57 (m_{c}, 1H), 3.64-3.85 (m, 2H), 3.96 (m_{c}, 1H), 4.35-4.51 (m, 2H), 4.58 (m_{c}, 1H), 6.94 (d, 1H), 6.99 (s, 1H), 7.10 (d, 1H), 7.28 (s, 1H), 7.32 (t, 1H), 7.38-7.46 (m, 2H).

### B. Assessment of the Physiological Activity

### Abbreviations Used:

AMP adenosine monophosphate
ATP adenosine triphosphate
BHI medium brain heart infusion medium
CoA coenzyme A
DMSO dimethyl sulfoxide
DTT dithiothreitol
EDTA ethylenediaminetetraacetic acid
KCl potassium chloride
KH₂PO₄ potassium dihydrogen phosphate
MgSO₄ magnesium sulfate
MIC minimum inhibitory concentration
MTP microtiter plate
NaCl sodium chloride
Na₂HPO₄ disodium hydrogen phosphate
NH₄Cl ammonium chloride
NTP nucleotide triphosphate
PBS phosphate-buffered saline
PCR polymerase chain reaction
PEG polyethylene glycol
PEP phosphoenolpyruvate
Tris tris[hydroxymethyl]aminomethane

The in vitro activity of the compounds of the invention can be shown in the following assays:

In Vitro Transcription-Translation with E. coli Extracts

In order to prepare an S30 extract logarithmically growing Escherichia coli MRE 600 (M. Müller; Freiburg University) are harvested, washed and employed as described for the in vitro transcription-translation test (Müller, M. and Blobel, G. Proc Natl Acad Sci USA (1984) 81, pp. 7421-7425).

1 μl of cAMP (11.25 mg/ml) per 50 μl of reaction mix are additionally added to the reaction mix of the in vitro transcription-translation test. The test mixture amounts to 105 μl, with 5 μl of the substance to be tested being provided in 5% DMSO. 1 μg/100 μl of mixture of the plasmid pBESTluc (Promega, Germany) are used as transcription template. After incubation at 30° C. for 60 min, 50 μl of luciferin solution (20 mM tricine, 2.67 mM MgSO₄, 0.1 mM EDTA, 33.3 mM DTT pH 7.8, 270 μM CoA, 470 μM luciferin, 530 μM ATP) are added, and the resulting bioluminescence is measured in a luminometer for 1 minute. The concentration of an inhibitor which leads to a 50% inhibition of the translation of firefly luciferase is reported as the IC₅₀.

In Vitro Transcription-Translation with S. aureus Extracts

Construction of an S. aureus Luciferase Reporter Plasmid

In order to construct a reporter plasmid which can be used in an in vitro transcription-translation assay from S. aureus the plasmid pBESTluc (Promega Corporation, USA) is used. The E. coli tac promoter present in this plasmid in front of the firefly luciferase is replaced with the capA1 promoter with corresponding Shine-Dalgarno sequence from S. aureus. The primers CAPF or 5′-CGGCCAAGCTTACTCGGATCCAGAGTTTGCAAAATATACAGGGGATT-ATATATAATGGAAAACAAGAAAGGAAAATAGGAGGTTTATATGGAAGACGCCA-3′ and CAPRev 5′-GTCATCGTCGGGAAGACCTG-3′ are used for this. The primer CAPFor contains the capA1 promoter, the ribosome binding site and the 5′ region of the luciferase gene. After PCR using pBESTluc as template it is possible to isolate a PCR product which contains the firefly luciferase gene with the fused capA1 promoter. This is, after restriction with ClaI and HindIII, ligated into the vector pBESTluc which has likewise been digested with ClaI and HindIII. The resulting plasmid p1a can be replicated in E. coli and be used as template in the S. aureus in vitro transcription-translation test.

Preparation of S30 Extracts from S. aureus

Six litres of BHI medium are inoculated with a 250 ml overnight culture of an S. aureus strain and allowed to grow at 37° C. up to an OD600 nm of 2-4. The cells are harvested by centrifugation and washed in 500 ml of cold buffer A (10 mM Tris acetate, pH 8.0, 14 mM magnesium acetate, 1 mM DTT, 1 M KCl). After renewed centrifugation, the cells are washed in 250 ml of cold buffer A with 50 mM KCl, and the resulting pellets are frozen at −20° C. for 60 min. The pellets are thawed on ice in 30 to 60 min and taken up to a total volume of 99 ml in buffer B (10 mM Tris acetate, pH 8.0, 20 mM magnesium acetate, 1 mM DTT, 50 mM KCl). 1.5 ml portions of lysostaphin (0.8 mg/ml) in buffer B are provided in 3 precooled centrifuge cups and each mixed with 33 ml of the cell suspension. The samples are incubated at 37° C., for 45 to 60 min shaking occasionally, before 150 μl of a 0.5 M DTT solution are added. The lysed cells are centrifuged at 30 000×g and 4° C. for 30 min. The cell pellet is taken up in buffer B and then centrifuged again under the same conditions, and the collected supernatants are combined. The supernatants are centrifuged again under the same conditions, and 0.25 volumes of buffer C (670 mM Tris acetate, pH 8.0, 20 mM magnesium acetate, 7 mM Na₃ phosphoenolpyruvate, 7 mM DTT, 5.5 mM ATP, 70 μM amino acids (complete from Promega), 75 μg of pyruvate kinase (Sigma, Germany))/ml are added to the upper ⅔ of the supernatant. The samples are incubated at 37° C. for 30 min. The supernatants are dialysed against 2 l of dialysis buffer (10 mM Tris acetate, pH 8.0, 14 mM magnesium acetate, 1 mM DTT, 60 mM potassium acetate) in a dialysis tube with a 3500 Da cut-off with one buffer change at 4° C. overnight. The dialysate is concentrated to a protein concentration of about 10 mg/ml by covering the dialysis tube with cold PEG 8000 powder (Sigma, Germany) at 4° C. The S30 extracts can be stored in aliquots at −70° C.

Determination of the IC₅₀ in the S. aureus In Vitro Transcription-Translation Assay

The inhibition of protein biosynthesis of the compounds can be shown in an in vitro transcription-translation assay. The assay is based on the cell-free transcription and translation of firefly luciferase using the reporter plasmid p1a as template and cell-free S30 extracts obtained from S. aureus. The activity of the resulting luciferase can be detected by luminescence measurement.

The amount of S30 extract or plasmid p1a to be employed must be tested anew for each preparation in order to ensure an optimal concentration in the test. 3 μl of the substance to be tested, dissolved in 5% DMSO, are provided in an MTP. Subsequently 10 μl of a suitably concentrated plasmid solution p1a are added. Subsequently 46 μl of a mixture of 23 μl of premix (500 mM potassium acetate, 87.5 mM Tris acetate, pH 8.0, 67.5 mM ammonium acetate, 5 mM DTT, 50 μg of folic acid/ml, 87.5 mg of PEG 8000 /ml, 5 mM ATP, 1.25 mM each NTP, 20 μM each amino acid, 50 mM PEP (Na₃ salt), 2.5 mM cAMP, 250 μg of each E. coli tRNA/ml) and 23 μl of a suitable amount of S. aureus S30 extract are added and mixed. After incubation at 30° C. for 60 min, 50 μl of luciferin solution (20 mM tricine, 2.67 mM MgSO₄, 0.1 mM EDTA, 33.3 mM DTT pH 7.8, 270 μM CoA, 470 μM luciferin, 530 μM ATP) are added, and the resulting bioluminescence is measured in a luminometer for 1 min. The concentration of an inhibitor which leads to a 50% inhibition of the translation of firefly luciferase is reported as the IC₅₀

Determination of the Minimum Inhibitory Concentration (MIC)

The minimum inhibitory concentration (MIC) is the minimum concentration of an antibiotic with which the growth of a test microbe is inhibited over 18-24 h. The inhibitor concentration can thereby be determined by standard microbiological methods (see, for example, The National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The MIC of the compounds of the invention is determined in the liquid dilution test on the 96-well microtiter plate scale. The bacterial microbes are cultivated in a minimal medium (18.5 mM Na₂HPO₄, 5.7 mM KH₂PO₄, 9.3 mM NH₄Cl, 2.8 mM MgSO₄, 17.1 mM NaCl, 0.033 μg/ml of thiamine hydrochloride, 1.2 μg/ml of nicotinic acid, 0.003 μg/ml of biotin, 1% glucose, 25 μg/ml of each proteinogenic amino acid with the exception of phenylalanine; [H.-P. Kroll; unpublished]) with addition of 0.4% BH broth (test medium). In the case of Enterococcus faecium L4001, heat-inactivated fetal calf serum (FCS; GibcoBRL, Germany) is added to the test medium in a final concentration of 10%. Overnight cultures of the test microbes are diluted to an OD₅₇₈ of 0.001 (to 0.01 in the case of enterococci) in fresh test medium, and incubated 1:1 with dilutions of the test substances (1:2 dilution steps) in test medium (200 μl final volume). The cultures are incubated at 37° C. for 18-24 hours; enterococci in the presence of 5% CO₂.

The lowest substance concentration in each case at which no visible bacterial growth occurs any longer is defined as the MIC.

Alternative Method for Determining the Minimum Inhibitory Concentration (MIC)

The minimum inhibitory concentration (MIC) is the minimum concentration of an antibiotic with which the growth of a test microbe is inhibited over 18-24 h. The inhibitor concentration can thereby be determined by standard microbiological methods with modified medium in an agar dilution test (see, for example, The National Committee for Clinical Laboratory Standards. Methods for dilution anti-microbial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The bacterial microbes are cultivated on 1.5% agar plates which contain 20% defibrinated horse blood. The test microbes, which are incubated overnight on Columbia blood agar plates (Becton-Dickinson), are diluted in PBS, adjusted to a microbe count of about 5×10⁵ microbes/ml and placed dropwise (1-3 μl) on test plates. The test substances comprise different dilutions of the test substances (1:2 dilution steps). The cultures are incubated at 37° C. in the presence of 5% CO₂ for 18-24 hours.

The lowest substance concentration in each case at which no visible bacterial growth occurs any longer is defined as the MIC and is reported in μg/ml.

TABLE A
(with comparative example biphenomycin B)

| | | MIC | IC₅₀ |
|---|---|---|---|
| | MIC | S. aureus | S. aureus 133 |
| Ex. No. | S. aureus 133 | T17 | Translation |
| 1 | 2.0 | 2.0 | 0.35 |
| 5 | 2.0 | 2.0 | 0.7 |
| 7 | 2.0 | 2.0 | 0.2 |
| Biphenomycin | <0.03 | >32 | 1.5 |
| B | | | |

Concentration data: MIC in μg/ml; IC₅₀ in μM.

Systemic Infection with S. aureus 133

The suitability of the compounds of the invention for treating bacterial infections can be shown in various animal models. For this purpose, the animals are generally infected with a suitable virulent microbe and then treated with the compound to be tested, which is in a formulation which is adapted to the particular therapy model. The suitability of the compounds of the invention can be demonstrated specifically for the treatment of bacterial infections in a mouse sepsis model after infection with S. aureus.

For this purpose, S. aureus 133 cells are cultured overnight in BH broth (Oxoid, Germany). The overnight culture was diluted 1:100 in fresh BH broth and expanded for 3 hours. The bacteria which are in the logarithmic phase of growth are centrifuged and washed twice with a buffered physiological saline solution. A cell suspension in a saline solution with an extinction of 50 units is then adjusted in a photometer (Dr Lange LP 2W). After a dilution step (1:15), this suspension is mixed 1:1 with a 10% mucine suspension. 0.2 ml of this infection solution is administered i.p. per 20 g of mouse. This corresponds to a cell count of about 1-2×10⁶ microbes/mouse. The i.v. therapy takes place 30 minutes after the infection. Female CFW1 mice are used for the infection experiment. The survival of the animals is recorded over 6 days. The animal model is adjusted so that untreated animals die within 24 h after the infection. It was possible to demonstrate in this model a therapeutic activity of ED₁₀₀=1.25 mg/kg for the compound of Example 2.

Determination of the Spontaneous Resistance Rates to S. aureus

The spontaneous resistance rates for the compounds of the invention are determined as follows: the bacterial microbes are cultivated in 30 ml of a minimal medium (18.5 mM Na₂HPO₄, 5.7 mM KH₂PO₄, 9.3 mM NH₄Cl, 2.8 mM MgSO₄,17.1 mM NaCl, 0.033 μg/ml of thiamine hydrochloride, 1.2 μg/ml of nicotinic acid, 0.003 μg/ml of biotin, 1% glucose, 25 μg/ml of each proteinogenic amino acid with the addition of 0.4% BH broth) at 37° C. over-night, centrifuged at 6000×g for 10 min and resuspended in 2 ml of a phosphate-buffered physiological NaCl solution (about 2×10⁹ microbes/ml). 100 μl of this cell suspension, and 1:10 and 1:100 dilutions, respectively, are plated out on predried agar plates (1.5% agar, 20% defibrinated horse blood, or 1.5% agar, 20% bovine serum in 1/10 Müller-Hinton medium diluted with PBS) which contain the compound of the invention to be tested in a concentration equivalent to 5×MIC or 10×MIC, and incubated at 37° C. for 48 h. The resulting colonies (cfu) are counted.

Isolation of the Biphenomycin-Resistant S. aureus Strains RN4220Bi^{R} and T17

The S. aureus strain RN4220Bi^{R} is isolated in vitro. For this purpose, 100 μl portions of an S. aureus RN4220 cell suspension (about 1.2×10⁸ cfu/ml) are plated out on an antibiotic-free agar plate (18.5 mM Na₂HPO₄, 5.7 mM KH₂PO₄, 9.3 mM NH₄Cl, 2.8 mM MgSO₄, 17.1 mM NaCl, 0.033 μg/ml of thiamine hydrochloride, 1.2 μg/ml of nicotinic acid, 0.003 μg/ml of biotin, 1% glucose, 25 μg/ml of each proteinogenic amino acid with the addition of 0.4% BH broth and 1% agarose) and on an agar plate containing 2 μg/ml of biphenomycin B (10×MIC), and incubated at 37° C. overnight. Whereas about 1×07 cells grow on the antibiotic-free plate, about 100 colonies grow on the antibiotic-containing plate, corresponding to a resistance rate of 1×10⁻⁵. Some of the colonies grown on the antibiotic-containing plate are tested for the biphenomycin B MIC. One colony with a MIC of >50 μM is selected for further use, and the strain is referred to as RN4220Bi^{R}.

The S. aureus strain T17 is isolated in vivo. CFW1 mice are infected intraperitoneally with 4×10⁷ S. aureus 133 cells per mouse. 0.5 h after the infection, the animals are treated intravenously with 50 mg/kg of biphenomycin B. The kidneys are removed from the surviving animals on day 3 after the infection. After homogenization of the organs, the homogenates are plated out as described for RN4220Bi^{R} on antibiotic-free and antibiotic-containing agar plates and incubated at 37° C. overnight. About half the colonies isolated from the kidney show growth on the antibiotic-containing plates (2.2×10⁶ colonies), demonstrating the accumulation of biphenomycin B-resistant S. aureus cells in the kidney of the treated animals. About 20 of these colonies are tested for the biphenomycin B MIC, and a colony with a MIC of >50 μM is selected for further cultivation, and the strain is referred to as T17.

Exemplary Embodiments of Pharmaceutical Compositions

The compounds of the invention can be converted into pharmaceutical preparations in the following way:

Solution which can be Administered Intravenously:

Composition:

1 mg of the compound of Example 1, 15 g of polyethylene glycol 400 and 250 g of water for injection.

Preparation:

The compound of the invention is dissolved together with polyethylene glycol 400 in the water while stirring. The solution is sterilized by filtration (pore diameter 0.22 μm) and dispensed under aseptic conditions into heat-sterilized infusion bottles. The latter are closed with infusion stoppers and crimped caps.

1. A compound of formula 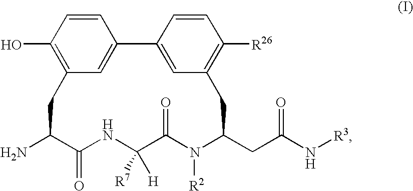
in which
R²⁶ represents hydrogen, halogen, hydroxy or methyl,
R⁷ represents a group of formula 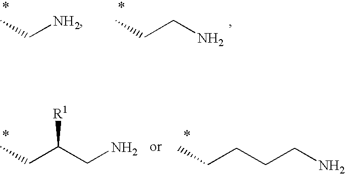
whereby
R¹ represents hydrogen or hydroxy,
* is the linkage site to the carbon atom,
R² represents hydrogen or methyl,
R³ represents a group of formula 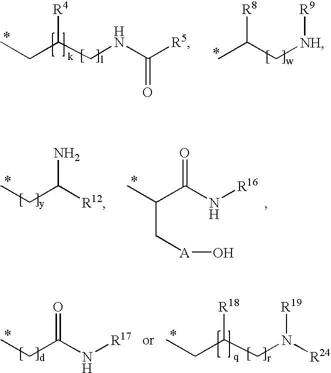
whereby
* is the linkage site to the nitrogen atom,
A represents a bond or phenyl,
R⁴ represents hydrogen, amino or hydroxy,
R⁵ represents a group of formula 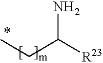
wherein
* is the linkage site to the carbon atom,
R²³ represents hydrogen or a group of formula *-(CH₂)ₙ—OH or *-(CH₂)ₒ—NH₂,
wherein
* is the linkage site to the carbon atom,
n and o independently of one another are a number 1, 2, 3 or 4,
m is a number 0 or 1,
R⁸ and R¹² independently of one another represent a group of formula *-CONHR¹⁴ or *-CH₂CONHR¹⁵
wherein
* is the linkage site to the carbon atom,
R¹⁴ and R¹⁵ independently of one another represent a group of formula 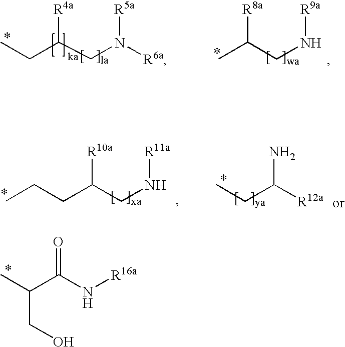
wherein
* is the linkage site to the nitrogen atom,
R^{4a} represents hydrogen, amino or hydroxy,
R^{5a} represents hydrogen, methyl or aminoethyl,
R^{6a} represents hydrogen or aminoethyl,
or
R^{5a} and R^{6a} together with the nitrogen atom to which they are bonded form a piperazine ring,
R^{8a} and R^{12a} independently of one another represent *-(CH₂)_{Z1a}—OH, *-(CH₂)_{Z2a}—NHR^{13a}, *-CONHR^{14a} or *-CH₂CONHR^{15a},
wherein
* is the linkage site to the carbon atom,
Z1a and Z2a independently of one another are a number 1, 2 or 3,
R^{13a} represents hydrogen or methyl,
and
R^{14a} and R^{15a} independently of one another represent a group of formula 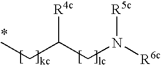
wherein
* is the linkage site to the nitrogen atom,
R^{4c} represents hydrogen, amino or hydroxy,
R^{5c} represents hydrogen, methyl or aminoethyl,
R^{6c} represents hydrogen or aminoethyl,
kc is a number 0 or 1,
and
lC is a number 1, 2, 3 or 4,
R^{9a} and R^{11a} independently of one another represent hydrogen or methyl,
R^{10a} represents amino or hydroxy,
R^{16a} represents a group of formula 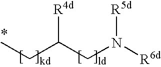
wherein
* is the linkage site to the nitrogen atom,
R^{4d} represents hydrogen, amino or hydroxy,
R^{5d} represents hydrogen, methyl or aminoethyl,
R^{6d} represents hydrogen or aminoethyl,
kd is a number 0 or 1,
and
ld is a number 1, 2, 3 or 4,
ka is a number 0 or 1,
and
la, wa, xa and ya independently of one another are a number 1, 2, 3 or 4,
R⁹ represents hydrogen, methyl, *-C(NH₂)═NH or a group of formula 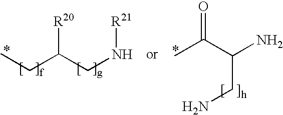
wherein
* is the linkage site to the nitrogen atom,
R²⁰ represents hydrogen or *-(CH₂)ᵢ—NHR²²,
wherein
R²² represents hydrogen or methyl,
and
i is a number 1, 2 or 3,
R²¹ represents hydrogen or methyl,
f is a number 0, 1, 2 or 3,
g is a number 1, 2 or 3,
and
h is a number 1, 2, 3 or 4,
R¹⁶ and R¹⁷ independently of one another represent a group of formula 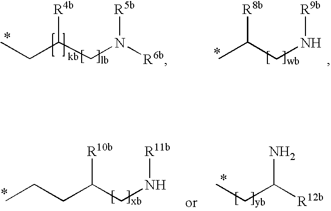
wherein
* is the linkage site to the nitrogen atom,
R^{4b} represents hydrogen, amino or hydroxy,
R^{5b} represents hydrogen, methyl or aminoethyl,
R^{6b} represents hydrogen or aminoethyl,
or
R^{5b} and R^{6b} together with the nitrogen atom to which they are bonded form a piperazine ring,
R^{8b} and R^{12b} independently of one another represent *-(CH₂)_{Z1b}—OH, *-(CH₂)_{z2b}—NHR^{13b}, *-CONHR^{4b} or *-CH₂CONHR^{15b},
wherein
* is the linkage site to the carbon atom,
R^{3b} represents hydrogen or methyl,
and
Z1b and Z2b independently of one another are a number 1, 2 or 3,
and
R^{4b} and R^{1b} independently of one another represent a group of formula 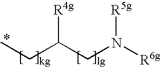
wherein
* is the linkage site to the nitrogen atom,
R^{4g} represents hydrogen, amino or hydroxy,
R^{5g} represents hydrogen, methyl or aminoethyl,
R^{6g} represents hydrogen or aminoethyl,
kg is a number 0 or 1,
and
lg is a number 1, 2, 3 or 4,
R^{9b} and R^{11b} independently of one another represent hydrogen or methyl,
R^{10b} represents amino or hydroxy,
kb is a number 0 or 1,
lb, wb, xb and yb independently of one another are a number 1, 2, 3 or 4,
R¹⁸ represents hydrogen, amino or hydroxy,
R¹⁹ represents hydrogen, methyl or a group of formula 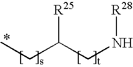
wherein
* is the linkage site to the nitrogen atom,
R²⁵ represents hydrogen or *-(CH₂)ᵤ—NHR
wherein
R²⁹ represents hydrogen or methyl,
and
u is a number 1, 2 or 3,
R²⁸ represents hydrogen or methyl,
s is a number 0, 1, 2 or 3,
and
t is a number 1, 2 or 3,
R²⁴ represents hydrogen or aminoethyl,
d is a number 1, 2 or 3,
k and q independently of one another are a number 0 or 1,
l, r, w and y independently of one another are a number 1, 2, 3 or 4, 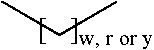
independently of one another may when w, r or y equals 3 carry a hydroxy group, or one of its salts, its solvates or the solvates of its salts.

2. The compound of claim 1, corresponding to formula 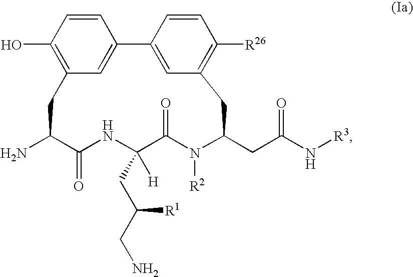
in which
R²⁶ represents hydrogen, halogen, hydroxy or methyl,
R¹ represents hydrogen or hydroxy,
R² represents hydrogen or methyl,
R³ is as defined in claim 1,
or one of its salts, its solvates or the solvates of its salts.

3. The compound of claim 1, wherein
R²⁶ represents hydrogen, chlorine, hydroxy or methyl.

4. The compound of claim 2, wherein
R²⁶ represents hydrogen or hydroxy,
R¹ represents hydrogen or hydroxy,
R² represents hydrogen,
R³ represents a group of formula 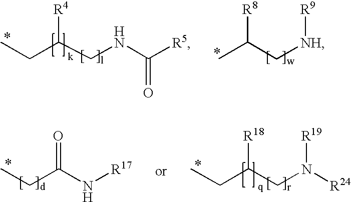
whereby
* is the linkage site to the nitrogen atom,
R⁴ represents hydrogen, amino or hydroxy,
R⁵ represents a group of formula 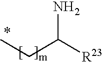
wherein
* is the linkage site to the carbon atom,
R²³ represents hydrogen or a group of formula *-(CH₂)ₙ—OH or *-(CH₂)ₒ—NH₂,
wherein
* is the linkage site to the carbon atom,
n and o independently of one another are a number 1, 2, 3 or 4,
m is a number 0 or 1,
R⁸ represents a group of formula *-CONHR¹⁴ or *-CH₂CONHR¹⁵,
wherein
* is the linkage site to the carbon atom,
R¹⁴ and R¹⁵ independently of one another represent a group of formula 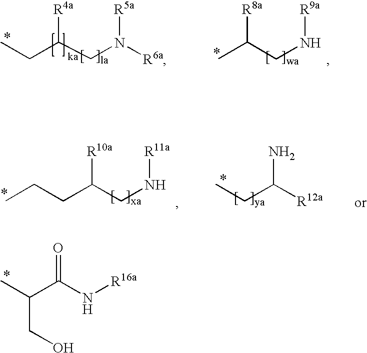
wherein
* is the linkage site to the nitrogen atom,
R^{4a} represents hydrogen, amino or hydroxy,
R^{5a} represents hydrogen, methyl or aminoethyl,
R^{6a} represents hydrogen or aminoethyl,
or
R^{5a} and R^{6a} together with the nitrogen atom to which they are bonded form a piperazine ring,
R^{8a} and R^{12a} independently of one another represent *-(CH₂)_{Z1a}—OH, *-(CH₂)_{Z2a}—NHR^{13a}, *-CONHR^{4a} or *-CH₂CONHR^{15a},
wherein
* is the linkage site to the carbon atom,
Z1a and Z2a independently of one another are a number 1, 2 or 3,
R^{13a} represents hydrogen or methyl,
and
R^{14a} and R^{15a} independently of one another represent a group of formula 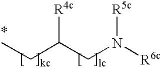
wherein
* is the linkage site to the nitrogen atom,
R^{4c} represents hydrogen, amino or hydroxy,
R^{5c} represents hydrogen, methyl or aminoethyl,
R^{6c} represents hydrogen or aminoethyl,
kc is a number 0 or 1,
and
lc is a number 1, 2, 3 or 4,
R^{9a} and R^{11a} independently of one another represent hydrogen or methyl,
R^{10a} represents amino or hydroxy,
R^{16a} represents a group of formula 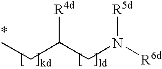
wherein
* is the linkage site to the nitrogen atom,
R^{4d} represents hydrogen, amino or hydroxy,
R^{5d} represents hydrogen, methyl or aminoethyl,
R^{6d} represents hydrogen or aminoethyl,
kd is a number 0 or 1,
and
ld is a number 1, 2, 3 or 4,
ka is a number 0 or 1,
and
la, wa, xa and ya independently of one another are a number 1, 2, 3 or 4,
R⁹ represents hydrogen, methyl, *-C(NH₂)═NH or a group of formula 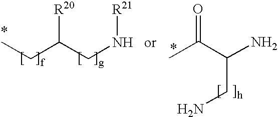
wherein
* is the linkage site to the nitrogen atom,
R²⁰ represents hydrogen or *-(CH₂)ᵢ—NHR²²
wherein
R²² represents hydrogen or methyl,
and
i is a number 1, 2 or 3,
R²¹ represents hydrogen or methyl,
f is a number 0, 1, 2 or 3,
g is a number 1, 2 or 3,
and
h is a number 1, 2, 3 or 4,
R¹⁷ represents a group of formula 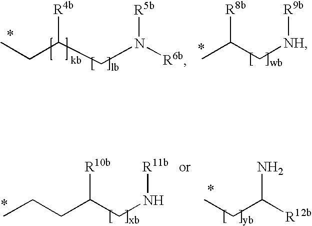
wherein
* is the linkage site to the nitrogen atom,
R^{4b} represents hydrogen, amino or hydroxy,
R^{5b} represents hydrogen, methyl or aminoethyl,
R^{6b} represents hydrogen or aminoethyl,
or
R^{5b} and R^{6b} together with the nitrogen atom to which they are bonded form a piperazine ring,
R^{8b} and R^{12b} independently of one another represent *-(CH₂)_{Z1b}—OH, *-(CH₂)_{Z2b}—NHR^{13b}, *-CONHR^{14b} or *-CH₂CONHR^{15b}
wherein
* is the linkage site to the carbon atom,
R^{13b} represents hydrogen or methyl,
and
Z1b and Z2b independently of one another are a number 1, 2 or 3,
and
R^{14b} and R^{15b} independently of one another represent a group of formula 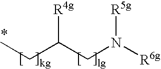
wherein
* is the linkage site to the nitrogen atom,
R^{4g} represents hydrogen, amino or hydroxy,
R^{5g} represents hydrogen, methyl or aminoethyl,
R^{6g} represents hydrogen or aminoethyl,
kg is a number 0 or 1,
and
lg is a number 1, 2, 3 or 4,
R^{9b} and R^{11b} independently of one another represent hydrogen or methyl,
R^{10b} represents amino or hydroxy,
kb is a number 0 or 1,
lb, wb, xb and yb independently of one another are a number 1, 2, 3 or 4,
R¹⁸ represents hydrogen, amino or hydroxy,
R¹⁹ represents hydrogen, methyl or a group of formula 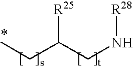
wherein
* is the linkage site to the nitrogen atom,
R²⁵ represents hydrogen or *-(CH₂)ᵤ—NHR²⁹
wherein
R²⁹ represents hydrogen or methyl
and
u is a number 1, 2 or 3,
R²⁸ represents hydrogen or methyl,
s is a number 0, 1, 2 or 3
and
t is a number 1, 2, or 3,
R²⁴ represents hydrogen or aminoethyl,
d is a number 1, 2, or 3
k and q independently of one another are a number 0 or 1,
l, r and w independently of one another are a number 1, 2, 3 or 4, 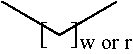
independently of one another may when w or r equals 3 carry a hydroxy group, or one of its salts, its solvates or the solvates of its salts.

5. The compound of claim 2, wherein
R²⁶ represents hydrogen or hydroxy,
R¹ represents hydrogen or hydroxy,
R² represents hydrogen,
R³ represents a group of formula 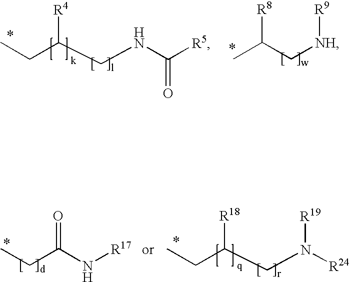
whereby
* is the linkage site to the nitrogen atom,
R⁴ represents hydrogen, amino or hydroxy,
R⁵ represents a group of formula 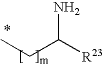
wherein
* is the linkage site to the carbon atom,
R²³ represents hydrogen or a group of formula *-(CH₂)ₙ—OH or *-(CH₂)ₒ—NH₂,
wherein
* is the linkage site to the carbon atom,
n and o independently of one another are a number 1, 2, 3 or 4,
m is a number 0 or 1,
R⁸ represents a group of formula *-CONHR⁴ or *-CH₂CONHR¹⁵,
wherein
* is the linkage site to the carbon atom,
R¹⁴ and R¹⁵ independently of one another represent a group of formula 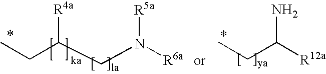
wherein
* is the linkage site to the nitrogen atom,
R^{4a} represents hydrogen, amino or hydroxy,
R^{5a} represents hydrogen,
R^{6a} represents hydrogen,
R^{2a} represents *-(CH₂)_{Z1a}—OH or *-CH₂CONHR^{15a},
wherein
* is the linkage site to the carbon atom,
Z1a is a number 1, 2 or 3,
and
R^{15a} represents a group of formula 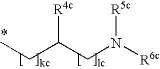
wherein
* is the linkage site to the nitrogen atom,
R^{4c} represents hydrogen, amino or hydroxy,
R^{5c} represents hydrogen,
R^{6c} represents hydrogen,
kc is a number 0 or 1,
and
lc is anumber 1, 2, 3 or 4,
ka is a number 0 or 1,
and
la and ya independently of one another are a number 1, 2, 3 or 4,
R⁹ represents hydrogen,
R¹⁷ represents a group of formula 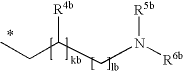
wherein
* is the linkage site to the nitrogen atom,
R^{4b} represents hydrogen, amino or hydroxy,
R^{5b} represents hydrogen,
R^{6b} represents hydrogen,
kb is a number 0 or 1,
lb is a number 1, 2, 3 or 4,
R¹⁸ represents hydrogen, amino or hydroxy
R¹⁹ represents hydrogen,
R²⁴ represents hydrogen,
d is a number 1, 2 or 3,
k and q independently of one another are a number 0 or 1,
l, r and w independently of one another are a number 1, 2, 3 or 4, 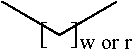
independently of one another may when w or r equals 3 carry a hydroxy group.
or one of its salts, its solvates or the solvates of its salts.

6. A method for preparing a compound of formula (I) of claim 1 or one of its salts, solvates or solvates of its salts, wherein
[A] a compound of formula 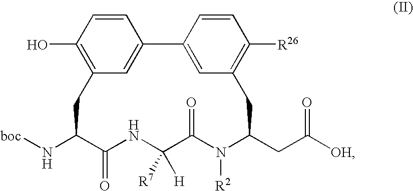
wherein R², R⁷ and R²⁶ have the meaning indicated in claim 1, and boc represents tert-butoxycarbonyl,
is reacted in a two-stage process firstly in the presence of one or more dehydrating reagents with a compound of formula
H₂NR³ (III),
wherein R³ has the meaning indicated in claim 1,
and subsequently with an acid, by hydrogenolysis or with an acid and by hydrogenolysis,
or
[B] a compound of formula 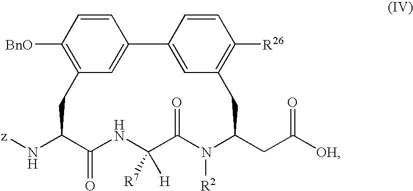
wherein R², R⁷ and R²⁶ have the meaning indicated in claim 1, and Z represents benzyloxycarbonyl,
is reacted in a two-stage process firstly in the presence of one or more dehydrating reagents with a compound of formula
H₂NR³ (III),
wherein R³ has the meaning indicated in claim 1,
and subsequently with an acid or by hydrogenolysis.

7. A method for preparing a compound of formula (I) of claim 1 or one of its solvates, wherein a salt of said compound or a solvate of a salt of said compound is converted into said compound by chromatography with the addition of a base.

8. The compound of claim 1 for the treatment, prophylaxis or treatment and prophylaxis of diseases.

9. A method for the production of a medicament for the treatment, prophylaxis or treatment and prophylaxis of diseases using a compound of claim 1.

10. A method for the production of a medicament for the treatment, prophylaxis or treatment and prophylaxis of bacterial diseases using a compound of claim 1.

11. A medicament comprising at least one compound of claim 1 in combination with at least one inert, non-toxic, pharmaceutically acceptable excipient.

12. The medicament of claim 11 for the treatment, prophylaxis or treatment and prophylaxis of bacterial infections.

13. A method for controlling bacterial infections in humans and animals by administering an antibacterially effective amount of at least one compound of claim 1.

14. A method for controlling bacterial infections in humans and animals by administering an antibacterially effective amount of at least one medicament of claim 11.
